(19) United States
(12) Patent Application Publication
(10) Pub. No.: US 2022/0296669 A1
(43) Pub. Date: Sep. 22, 2022

(54) NOVEL COMPOSITIONS AND USES THEREOF

(71) Applicant: COLZYX AB, Lund (SE)

(72) Inventors: Matthias MÖRGELIN, Anderslöv (SE); Suado Mohamed ABDILLAHI, Lund (SE)

(21) Appl. No.: 17/619,277

(22) PCT Filed: Jun. 26, 2020
(86) PCT No.: PCT/EP20/68047

§ 371 Date: Dec. 15, 2021

(30) Foreign Application Priority Data
Jun. 28, 2019 (GB) 1909298.0

(51) Int. Cl. A61K 38/01; A61L 2/28; A61P 31/00; A61P 33/00; A61L 27/24; A61F 15/00

(52) U.S. Cl. CPC A61K 38/014; A61L 2/28; A61P 31/00; A61P 33/00; A61L 27/24; A61F 15/008

(57) ABSTRACT

The present invention provides a composition comprising: (a) collagen VI or a polypeptide comprising or consisting of an amino acid sequence derived from collagen type VI or a fragment, variant, fusion or derivative thereof; and (b) polylysine. Also provided are pharmaceutical compositions and kits comprising the composition of the invention. Related aspects provide medical devices, implants, wound care products and materials for use in the same associated with the composition of the invention, and methods of their preparation. Also provided are methods and uses of the composition in the treatment and/or prevention of microbial infections and in wound care, and a method of killing microorganisms in vitro.

### FIELD OF INVENTION

The present invention relates to compositions comprising collagen VI polypeptides and derivatives thereof in combination with polylysine; medical devices, implants, wound care products, and kits comprising the same; and uses and medical uses thereof.

### Background

Skin is our major external defence system, in charge of protecting our inner body structures from invading microorganisms, and the adverse effects of the external environment. Adult skin is composed of three layers: epidermis or stratum corneum, mainly consisting of keratinocytes; dermis, the connective tissue rich in collagen and elastin; and hypodermis or subcutaneous layer, composed of fat tissue, which provides thermal isolation and mechanical protection to the body [1]. Wounds are superficial or deep injuries within the skin, which may form due to physicochemical or thermal damage. Acute wounds are defined by injured tissues that need a healing period over 8-12 weeks, (e.g., burns, chemical injuries, cuts). In contrast, chronic wounds are a fallout of diseases, such as venous or arterial vascular insufficiency, pressure necrosis, cancer, and diabetes [2,3]. They require longer healing time (weeks-months to years) and often fail to reach a normal healthy state, persisting in a pathological condition of inflammation [4]. Therefore, delayed or impaired wound healing poses a significant socio-economic burden on patients and health care systems worldwide, in terms of treatment costs and waste production [5].

Tissue regeneration after injury is an intricate process where devitalized cellular and tissue structures are replaced [6]. Insight into the carefully orchestrated biochemical and cellular events activated during skin repair is crucial to design appropriate wound dressings [1,7,8]. It comprises extensive changes in cell response as well as in extracellular matrix (ECM) composition. In general, wound repair is divided into different predictable and overlapping phases: haemostasis, inflammation, proliferation, followed by maturation and remodulation of the scar tissue [9-11]. Haemostasis is the immediate response of the body to an injury, in order to stop blood loss at the wound site, by means of fibrin cloths as temporary barriers [12]. Inflammation (from 24 h to 4-6 days) is mediated by neutrophils and macrophages [13], that sweep the wound bed from foreign particles and tissue debris. Cytokines and enzymes are released to stimulate fibroblasts and myofibroblasts macrophages [14], while the wound exudate provides the essential moisture for the recovery. In the proliferation phase epithelialization occurs and newly formed granulation tissue begins to fill the wound area, producing new ECM. Finally, during the remodeling phase, collagen-based cross-linking is responsible for a tight 3D network formation, increasing the tensile strength of the new tissue [12]. Importantly, the intimate relationship between cells and their surrounding framework is commonly regarded to play a pivotal role in regulating regenerative processes. Thus, it is pivotal to create appropriate biomaterials which are extensively loaded with biomolecules in order to ensure premium wound healing properties.

Following the first two phases of wound healing is a repairing phase, termed the proliferative phase. It begins 3 days after the injury and lasts for 2 weeks. After injury, fibroblasts and myofibroblasts proliferate in local wound milieu and are stimulated by TGFβ and PDGF, to migrate to the site of injury on the third day, where they proliferate profusely. Fibroblasts lay extracellular matrix of matrix proteins: hyaluronan, fibronectin, proteoglycans, and extended fibrillar networks consisting of type I/III/V collagen. This matrix helps support cell migration and repair process. Wound contraction, an important reparative process to approximate wound edges, takes place after which fibroblasts are eliminated [15].

Collagen is the most abundant mammalian protein, which provides mechanical strength to tissues and stimulates cell-adhesion and proliferation [16,17]. About thirty different types of collagen have been identified, displaying a triple-helical tertiary structure of polypeptide sequences, but only a few are used in the production of collagen-based biomaterials.

Collagen type VI forms complex and extensive beaded microfibrillar network in most connective tissues. The predominant form of collagen type VI is composed of three distinct polypeptide chains, α1(VI), α2(VI) and α3(VI), which form triple helical monomers. Inside the cell, the monomers assemble into dimers and tetramers that are secreted into the extracellular space [18]. There, the tetramers aggregate end-on-end to form microfibrils that become part of extended supramolecular matrix assemblies. More recently, three additional chains (α4, α5 and α6) were discovered, which may substitute for the α3-chain in some tissues [18]. In terms of structure, each α-chain is characterized by a short extended triple-helical region flanked by two large N- and C-terminal globular regions, which share homology with von Willebrand Factor type A domains (VWA) [19-22]. VWA is also responsible for protein-protein interaction in the ECM [19-22]. The α1(VI) and α2(VI) chains of collagen type VI contain one N-terminal (N1) and two C-terminal (C1 and C2) VWA domains, whereas the α3(VI) is much larger and comprises some ten N-terminal (N10-N1) VWA domains and two C-terminal VWA domains. Additionally, the α3(VI) chain has three C-terminal domains (C3-05) that share homology with salivary gland proteins, fibronectin type III repeats and the Kunitz family of serine protease inhibitors [23]. With its unique setup, collagen type VI provides strength, integrity and structure to wide range of tissues. It is also involved in other important biological processes such as apoptosis, autophagy, angiogenesis, fibrosis and tissue repair [24].

The inventors have previously shown that collagen VI peptides and derivatives thereof have antimicrobial properties (see WO 2017/125585, the content of which is hereby incorporated by reference), making them useful for applications relating to the killing or growth inhibition of bacteria that have developed resistance to conventional antibiotics, such as MRSA. They are also useful in the treatment of microbial infections and wounds, for example when coated to the surface of a medical device or other such material.

High biocompatibility and biodegradability by endogenous collagenases make collagen ideal for biomedical applications [25,26]. During wound healing, fibroblasts produce collagen molecules that aggregate to form fibrils with diameter in the range of 10-500 nm. This fibrous network facilitates cell migration to the wounded site, actively supporting tissue repair [27]. Thanks to a facile chemical functionalization of the protein structure, various dressing architectures have been exploited. Collagen-based wound dressings, either in forms of hydrogels, electrospun fibers, or nanocrystal-containing scaffolds, have been applied to cover burn wounds, treat ulcers [28-31], reduce tissue contraction and scarring, and increase epithelialization rate [32]. Collagen sponges and fibrous membranes were found particularly promising, due to their wet strength that allows suturing to soft tissues and provides a template for new tissue growth. The multifunctional platform showed anti-bacterial and anti-inflammatory properties, while retaining a favourable topography for cell proliferation, thus accelerating healing and wound closure. Despite their rather extensive usage as biomaterials for scaffold design, collagen scaffolds remain sustainable materials with high engineering potential that is as yet unexplored [33-35].

Given the multiple cellular mechanisms involved in the skin wound healing process and the interplay of several external factors, the choice of suitable dressing materials is compelling. Specifically, for biodegradable natural materials, their degradation needs to follow the dynamics of the wound repair, guaranteeing the physiological healing evolution, and releasing active principles when needed [6]. To address this issue several wound dressings are designed to achieve the highest level of biomimicry by recapitulating the native extracellular matrix biological and physico-chemical features. In particular, biological dressings based on native collagen networks are used to improve the wound microenvironment and thus favour the regeneration of new tissue. The rationale behind the use of these materials is the approach that they present inherent natural cell-recognition domains and thus stimulate fibroblast and keratinocyte adhesion and proliferation in the wound bed [36]. In addition, such natural fibrillar collagen scaffolds are thought to provide guiding ridges for invading cells and thus may promote structured dermal wound healing.

Metallic or composite endoprostheses are engineered biomaterial systems that are placed in the human body in order to maintain or replace functions that have been impaired due to infection, disease, accident, or ageing [37-42]. They are used largely in orthopaedic joint replacements, arthroplasty, orthopaedic fixations, dental implants, prosthetic cardiac devices, and coronary stents. The use of biomaterials has been steadily increasing worldwide over the past decades. This development is fueled by ever-increasing ageing of populations in developed countries and the desire of the patients to maintain the same level of activity and quality of life [43]. Consequently, the demand for high-performance implantable biomaterials that can address unique challenges in cardiology, vascular therapy, orthopedics, trauma, spine, dental and wound care has also been increasing steadily.

The diversity and functionality of available biomaterials have also experienced substantial growth, with a wide variety of synthetic, natural and hybrid materials currently on the market, such as metals, ceramics, or polymers [44-47]. Thereby, metallic materials can be made of stainless steel, titanium or alloys of titanium, cobalt-chromium, magnesium, tantalum, or niobium [48]. Titanium and its alloys are widely used in metallic and composite biomaterials, comprising 70%-80% of all used materials [49-53]. Due to their excellent mechanical properties, fatigue strength, corrosion resistance, and biocompatibility, these materials are used in load-bearing areas such as orthodontics, orthopedics, and gastroenterology as well as for cardiovascular and reconstructive purposes [54-56]. Commercially pure titanium (CP Ti) and Ti₆Al₄V alloys were established in the 1950s and offer excellent physical and chemical properties for dental applications, joints, orthopedic trauma and reconstruction surgery and attachment systems [57]. In addition, in recent years, the group of shape memory alloys like nickel-titanium (Nitinol) have risen in importance [58].

The clinical survival and success rates of implanted biomaterials are largely determined by the long-term stability in the patient. Primary implant stability at placement is a mechanical phenomenon that depends on local bone quality and quantity, the type of implant and the surgical technique. The long-term stability of the implant is affected by bone formation and remodeling at the host-biomaterial interface and in the surrounding bone tissue [59]. Even though today's success rates are relatively high, there is a constant need to improve structural and functional fusion of the implant surface with the surrounding bone tissue of the host.

There therefore remains a need for development of improved compositions that are capable of achieving improved binding of peptides such as collagen VI to materials, such as medical devices, implants, wound care products and materials for use in the same. Improved binding can provide improved delivery of the peptide, and in turn improved performance of the medical device, implant, wound care product or material for use in the same which it is associated with.

Here, the inventors have shown for the first time that the use of polylysine greatly enhances binding of collagen VI molecules, such as in the surface coating of biological collagen scaffolds with native bioactive collagen VI molecules and in the surface coating of titanium containing materials. This effect will result in a pronounced potential to provide a versatile, multifunctional, and appropriate extracellular environment, able to actively contrast the onset of infections and inflammation, while promoting tissue regeneration and scar remodeling, and consequently deliver the desired enhancement in biocompatibility.

### SUMMARY OF THE INVENTION

A first aspect of the invention provides a composition comprising:
(a) collagen type VI or a polypeptide comprising or consisting of an amino acid sequence derived from collagen type VI, or a fragment, variant, fusion or derivative thereof, or a fusion of said fragment, variant of derivative thereof; and
(b) polylysine.

It will be appreciated by persons skilled in the art that the collagen type VI may be from a human or non-human source. For example, the collagen type VI may be derived (directly or indirectly) from a non-human mammal, such as an ape (e.g. chimpanzee, bonobo, gorilla, gibbon and orangutan), monkey (e.g. macaque, baboon and colobus), rodent (e.g. mouse, rat) or ungulates (e.g. pig, horse and cow). The collagen VI may also be derived from birds, e.g. chicken (Gallus gallus).

Thus, by “collagen type VI” (also “collagen VI”) we include naturally occurring human collagen type VI, collagen type VI monomers, dimers and tetramers, collagen type VI microfibrils, and homologues thereof, such as bovine collagen type VI.

Collagen type VI is typically comprised of each of three collagen VI peptide chains (α1(VI)), α2(VI) and α3(VI)). In some cases, the α3(VI) chain may be substituted for the α4(VI), α5(VI) or α6(VI) chain.

The sequences of the different collagen VI alpha chains are publicly available, such as at UniProt (https://www.uniprot.org/). Details of the UniProt ID for each alpha chain and the individual pages at UniProt are as follows:

Alpha-1: https://www.uniprot.org/uniprot/P12109

Alpha-2: https://www.uniprot.org/uniprot/P12110

Alpha-3: https://www.uniprot.org/uniprot/P12111

Alpha-4: https://www.uniprot.org/uniprot/A2AX52

Alpha-5: https://www.uniprot.org/uniprot/A8TX70

Alpha-6: https://www.uniprot.org/uniprot/A6NMZ7

Therefore, in certain embodiments, the collagen VI of the composition of the invention comprises or consists of any three amino acid chains selected from the group consisting of α1(VI), α2(VI), α3(VI), α4(VI), α5(VI) and α6(VI). In one embodiment the collagen VI comprises or consists of an α1(VI)) chain, an α2(VI) chain and an α3(VI) chain.

In one embodiment, the polypeptide of the composition of the first aspect is or is derived from the α1, α2 and/or α3 chain of collagen type VI.

In one embodiment, the polypeptide of the composition of the first aspect is or is derived from a collagen VI α3(VI) chain.

In another embodiment, the collagen VI may comprise one α1(VI) chain, one α2(VI) chain and further comprises a third chain that is either an α3, α4, α5 or α6 chain.

In certain embodiments, the collagen VI is, or is a peptide derived from, human collagen VI. In an alternative embodiment the collagen VI is, or is a peptide derived, from bovine collagen VI.

In one embodiment, the polypeptide comprising or consisting of an amino acid sequence derived from collagen type VI, or a fragment, variant, fusion or derivative thereof, or a fusion of said fragment, variant of derivative thereof, is derived from the collagen VI α3 chain.

In one embodiment, the polypeptide comprising or consisting of an amino acid sequence derived from collagen type VI, or a fragment, variant, fusion or derivative thereof, or a fusion of said fragment, variant of derivative thereof, comprises or consists of one or more polypeptides selected from the group consisting of α1(VI), α2(VI) and α3(VI).

In one embodiment, the composition comprises one or more polypeptides selected from the group consisting of: collagen VI, collagen VI α1 chain, collagen VI α2 chain, collagen VI α3 chain, GVR28, FYL25, FFL25, VTT30, SFV33.

By an amino acid sequence “derived from” collagen type VI we include amino acid sequences found within the amino acid sequence of a naturally occurring collagen type VI protein. In particular, we include amino acid sequences that comprise at least five contiguous amino acids from the sequence of a naturally occurring collagen type VI. For example, in one embodiment the amino acid sequence may contain at least 5 contiguous amino acids from collagen type VI, for example at least 6, 7, 8, 9, 10, 11, 12, 13, 14, 15, 16, 17, 18, 19, 20, 21, 22, 23, 24, 25, 30, 40, or 50 contiguous amino acids from collagen type VI. Thus, the amino acid sequence derived from collagen type VI corresponds to a fragment of collagen type VI.

In one embodiment, the collagen VI or polypeptide, fragment, variant, fusion or derivative thereof of collagen VI is capable of killing or attenuating the growth of microorganisms.

The antimicrobial properties of collagen VI and polypeptides comprising an amino acid sequence derived from collagen VI, such as GVR28, FYL25, FFL25, VTT30, and SFV33, are described in WO 2017/125585, the content of which is incorporated herein by reference.

By “capable of killing or attenuating the growth of microorganisms” we include collagen VI and polypeptides with antimicrobial activity. The antimicrobial activity may be in whole or in part, and may be dose dependent. This may be demonstrated by, for example, radial diffusion assays.

The microorganisms against which the polypeptides of the invention are efficacious may be selected from the group consisting of bacteria, mycoplasmas, yeasts, fungi and viruses.

In one embodiment, the collagen VI or polypeptide of the composition of the first aspect is capable of binding to the membrane of the microorganism. In another embodiment, the collagen VI or polypeptide may have affinity for negatively charged surfaces, for example a bacterial membrane. This affinity may be tested by, for example, affinity to heparin, wherein higher affinity to heparin indicates higher affinity to negatively charged surfaces.

Advantageously, the affinity or binding capability of the collagen VI or polypeptide is comparable to or greater than that of LL-37. Thus, in one embodiment, the collagen VI or polypeptide is capable of exhibiting an antimicrobial effect greater than or equal to that of LL-37 LLGDFFRKSKEKIGKEFKRIVQRIKDFLRNLVPRTES; SEQ ID NO: 24).

In one embodiment, the collagen VI or polypeptide is capable of causing structural alterations to the microorganism, including, for example, membrane perturbations, blebbing or exudation of cytoplasmic constituents.

Thus, the collagen VI or polypeptide of the composition may be capable of causing disruption of the membrane of the microorganisms. This can, for example, be quantified through microscopy studies, such as electron microscopy or fluorescence microscopy, studying the uptake of fluorescent molecules by the microorganisms.

In a further embodiment, the collagen VI or polypeptide of the composition may be capable of promoting wound closure and/or wound healing.

By “promoting wound closure” and/or “wound healing” we include aiding the healing process of the wound, for example by accelerating healing. For example, the wound care product may be capable of enhancing epithelial regeneration and/or healing of wound epithelia and/or wound stroma. In one embodiment, the wound care product may be capable of enhancing the proliferation of epithelial and/or stromal cells through a non-lytic mechanism. The wound closing capability may be quantified by, for example, cell scratch experiments.

Thus, the collagen VI or polypeptide of the composition may have a role in wound care by promoting wound closure/healing and/or by preventing infection of a wound.

The wounds to be treated by the composition of the first aspect may be extracorporeal (i.e. surface wounds of the skin and underlying tissue) and/or intracorporeal (such as internal wounds due to organ transplantation or removal of tissue/parts of organs, e.g. following colon surgery).

It will be appreciated by persons skilled in the art that the composition of the first aspect may exert an antimicrobial effect against Gram-positive and/or Gram-negative bacteria. For example, the microorganisms may be bacteria selected from the group consisting of: Pseudomonas aeruginosa, Staphylococcus aureus, Escherichia coli, group A Streptococcus (e.g. Streptococcus pyogenes), group B Streptococcus e.g. Streptococcus agalactiae), group C Streptococcus (e.g. Streptococcus dysgalactiae), group D Streptococcus (e.g. Entero-coccus faecalis), group F Streptococcus (e.g. Streptococcus anginosus), group G Streptococcus (e.g. Streptococcus dysgalactiae equisimilis), alpha-hemolytic Streptococcus (e.g. Streptococcus viridans, Streptococcus pneumoniae), Streptococcus bovis, Streptococcus mitis, Streptococcus anginosus, Streptococcus sanguinis, Streptococcus suis, Streptococcus mutans, Moraxella catarrhalis, Non-typeable Haemophilus influenzae (NTHi), Haemophilus influenzae b (Hib), Actinomyces naeslundii, Fusobacterium nucleatum, Prevotella intermedia, Klebsiella pneumoniae, Enterococcus cloacae, Enterococcus faecalis, Staphylococcus epidermidis, multi-resistant Pseudomonas aeruginosa (MRPA), multi-resistant Staphylococcus aureus (MRSA), multi-resistant Escherichia coli (MREC), multi-resistant Staphylococcus epidermidis (MRSE) and multi-resistant Klebsiella pneumoniae (MRKP).

In one embodiment the microorganisms are bacteria which are resistant to one or more conventional antibiotic agents.

By “conventional antibiotic agent” we include known agents that are capable of killing or attenuating the growth of microorganisms, for example natural and synthetic penicillins and cephalosporins, sulphonamides, erythromycin, kanomycin, tetracycline, chloramphenicol, rifampicin and including gentamicin, ampicillin, benzypenicillin, benethamine penicillin, benzathine penicillin, phenethicillin, phenoxy-methyl penicillin, procaine penicillin, cloxacillin, flucloxacillin, methicillin sodium, amoxicillin, bacampicillin hydrochloride, ciclacillin, mezlocillin, pivampicillin, talampicillin hydrochloride, carfecillin sodium, piperacillin, ticarcillin, mecillinam, pirmecillinan, cefaclor, cefadroxil, cefotaxime, cefoxitin, cefsulodin sodium, ceftazidime, ceftizoxime, cefuroxime, cephalexin, cephalothin, cephamandole, cephazolin, cephradine, latamoxef disodium, aztreonam, chlortetracycline hydrochloride, clomocycline sodium, demeclocydine hydrochloride, doxycycline, lymecycline, minocycline, oxytetracycline, amikacin, framycetin sulphate, neomycin sulphate, netilmicin, tobramycin, colistin, sodium fusidate, polymyxin B sulphate, spectinomycin, vancomycin, calcium sulphaloxate, sulfametopyrazine, sulphadiazine, sulphadimidine, sulphaguanidine, sulphaurea, capreomycin, metronidazole, tinidazole, cinoxacin, ciprofloxacin, nitrofurantoin, hexamine, streptomycin, carbenicillin, colistimethate, polymyxin B, furazolidone, nalidixic acid, trimethoprim-sulfamethox-azole, clindamycin, lincomycin, cycloserine, isoniazid, ethambutol, ethionamide, pyrazinamide and the like; anti-fungal agents, for example miconazole, ketoconazole, itraconazole, fluconazole, amphotericin, flucytosine, griseofulvin, natamycin, nystatin, and the like; and anti-viral agents such as acyclovir, AZT, ddl, amantadine hydrochloride, inosine pranobex, vidarabine, and the like.

Thus, in one embodiment, the microorganism is selected from the group consisting of: multidrug-resistant Staphylococcus aureus (MRSA) (methicillin resistant Staphylococcus aureus), multidrug-resistant Pseudomonas aeruginosa (M RPA), multidrug-resistant Escherichia coli (MREC), multidrug-resistant Staphylococcus epidermidis (MRSE) and multidrug-resistant Klebsiella pneumoniae (MRKP).

Advantageously, the composition according to the first aspect of the invention exhibits selective toxicity to microbial agents. By ‘selective’ we mean the polypeptide of the composition is preferentially toxic to one or more microorganisms (such as bacteria, mycoplasmas, yeasts, fungi and/or viruses) compared to mammalian, e.g. human, host cells. For example, the toxicity of the polypeptide of the composition to a target microorganism is at least two-fold greater than the toxicity of that composition to mammalian cells, more preferably at least three-fold, at least four-fold, at least five-fold, at least six-fold, at least eight-fold, at least ten-fold, at least fifteen-fold or at least twenty fold.

Conveniently, the collagen VI or polypeptide of the composition of the first aspect is substantially non-toxic to mammalian, e.g. human, cells.

For example, the collagen VI or polypeptide of the composition of the first aspect may not exhibit cytotoxicity to erythrocytes or monocytes at concentrations at concentrations which can be used to kill microorganisms such as bacteria. In one embodiment the polypeptide of the composition does not exhibit cytotoxicity at a concentration of up to 30 μM, or alternatively at a concentration of up to 50 μM.

In this way, when the compounds are used to treat microbial infections, for example, dosing regimens can be selected such that microbial cells are destroyed with minimal damage to healthy host tissue. Thus, the collagen VI or polypeptide of the composition may exhibit a ‘therapeutic window’.

In one embodiment, the collagen VI or polypeptide of the composition is capable of exerting an anti-endotoxic effect.

By “anti-endotoxic effect” we include polypeptides which counteract the effects induced by endotoxins. For example, in one embodiment the collagen VI or polypeptide of the composition is capable of suppressing, at least in part, LPS induction of nitrite.

In one embodiment, the polypeptide of the composition is derived from or shows amino acid sequence homology to a VWA domain, for example a globular VWA domain. Thus, the polypeptide of the composition may comprise or consist of an amino acid sequence which corresponds to at least five (for example, at least 10, 15, 20 or more) contiguous amino acids of a VWA domain, or an amino acid sequence which has at least 70% (for example at least 80%, 90% or 95%) identity with such as sequence.

In a further embodiment, the polypeptide of the composition may comprise or consist of an intact VWA domain.

By “VWA domain” we include the type A domains of von Willebrand factor, and domains showing homology to the type A domains of von Willebrand factor, as well as VWA-domain containing regions.

In one embodiment, the polypeptide of the composition is derived from the α3 chain of collagen type VI. Thus, the polypeptide of the composition may be derived from the α3N or α3C regions. For example, the polypeptide of the composition may be or may be derived from the N2, N3 or C1 domain of the α3 chain of collagen type VI.

In an alternative embodiment, the polypeptide of the composition is derived from the α4 chain of collagen type VI.

In another alternative embodiment, the polypeptide of the composition is derived from the α5 chain of collagen type VI.

In a further alternative embodiment, the polypeptide of the composition is derived from the α6 chain of collagen type VI.

In a still further alternative embodiment, the polypeptide of the composition is derived from the α2 chain of collagen type VI, for example from the α2N region.

It will be appreciated by persons skilled in the art that the collagen VI or polypeptide of the composition of the first aspect may have cationic residues on their surface, or cationic sequence motifs therein.

Thus, in one embodiment, the collagen VI or polypeptide of the composition has a net positive charge. For example, the polypeptide may have a charge ranging from between +2 to +9.

In a further embodiment, the collagen VI or polypeptide of the composition has at least 30% hydrophobic residues.

In a still further embodiment, the collagen VI or polypeptide of the composition may have an amphipathic structure.

Exemplary collagen VI polypeptides of the composition of the first aspect of the invention comprise or consist of the amino acid sequence of any one of SEQ ID NOs: 1 to 23 (as shown in Table 1) or a fragment, variant, fusion or derivative thereof, or a fusion of said fragment, variant or derivative thereof, which retains an antimicrobial activity of any one of SEQ ID NOs:1 to 23.

TABLE 1
Exemplary polypeptides of the composition of the invention

| Peptide | Sequence | MW | Charge |
|---|---|---|---|
| GVR28 | GVRPDGFAHIRDFVSRIVRRLNIGPSKV [SEQ ID NO: 1] | 3,163 | 4 |
| FYL25 | FYLKTYRSQAPVLDAIRRLRLRGGS [SEQ ID NO: 2] | 2,937 | 5 |
| FFL25 | FFLKDFSTKRQIIDAINKVVYKGGR [SEQ ID NO: 3] | 2,944 | 4 |
| VTT30 | VTTEIRFADSKRKSVLLDKIKNLQVALTSK [SEQ ID NO: 4] | 3,403 | 4 |
| SFV33 | SFVARNTFKRVRNGFLMRKVAVFFSNTPTRASP [SEQ ID NO: 5] | 3,803 | 7 |
| DVN32 | DVNVFAIGVEDADEGALKEIASEPLNMHMFNL [SEQ ID NO: 6] | 3,490 | −6 |
| KPE20 | KPEILNLVKRMKIKTGKALN [SEQ ID NO: 7] | 2,295 | 5 |
| GFA20 | GFAHIRDFVSRIVRRLNIGP [SEQ ID NO: 8] | 2,324 | 5 |
| QAP20 | QAPVLDAIRRLRLRGGSPLN [SEQ ID NO: 9] | 2,203 | 3 |
| KGF20 | KGFESKVDAILNRISQMHRV [SEQ ID NQ: 10] | 2,329 | 3 |
| RKV20 | RKVAVFFSNTPTRASPQLRE [SEQ ID NO: 11] | 2,305 | 2 |
| VAA20 | VAAKPVATKMAVRPPVAVKP [SEQ ID NO: 12] | 2,031 | 3 |
| AAK20 | AAKPVATKPEVPRPQAAKPA [SEQ ID NO: 13] | 2,027 | 4 |
| TTK20 | TTKPVTTTKPVTTTTKPVTT [SEQ ID NO: 14] | 2,103 | 3 |
| AAA76 | AAAKPAPAKPVAAKPVATKMATVRPPVAVKPATAAKPVAAKPA | 7,324 | 13 |
| | AVRPPAAAAAKPVATKPEVPRPQAAKPAATKPA [SEQ ID NO: 15] | | |
| TSS36 | TSSPTSNPVTTTKPVTTTKPVTTTKPVTTTKPVT [SEQ ID NO: 16] | 3,703 | 4 |
| YDR20 | YDRLIKESRRQKTRVFAVVI [SEQ ID NO: 17] | 2 473 | 4 |
| EQN20 | EQNFHKARRFVEQVARRLTL [SEQ ID NO: 18] | 2 499 | 3 |
| VVH20 | VVHAINAIVRSPRGGARRHA [SEQ ID NO: 19] | 2 137 | 4 |
| LRL20 | LRLKPYGALVDKVKSFTKRF [SEQ ID NO: 20] | 2 367 | 5 |
| FTK20 | FTKRFIDNLRDRYYRCDRNL [SEQ ID NO: 21] | 2 665 | 3 |
| RDA20 | RDALKSSVDAVKYFGKGTYT [SEQ ID NO: 22] | 2 206 | 2 |
| TKR20 | TKRFAKRLAERFLTAGRTDP [SEQ ID NO: 23] | 2 335 | 4 |

For example, the polypeptide of the composition of the invention may comprise or consist of an amino acid sequence selected from the group consisting of SEQ ID NOs: 1 to 5:

[SEQ ID NO: 1]
“GVR28”: GVRPDGFAHIRDFVSRIVRRLNIGPSKV
[SEQ ID NO: 2]
“FYL25”: FYLKTYRSQAPVLDAIRRLRLRGGS
[SEQ ID NO: 3]
“FFL25”: FFLKDFSTKRQIIDAINKVVYKGGR
[SEQ ID NO: 4]
“VTT30”: VTTEIRFADSKRKSVLLDKIKNLQVALTSK
[SEQ ID NO: 5]
“SFV33”: SFVARNTFKRVRNGFLMRKVAVFFSNTPTRASP

or a fragment, variant, fusion or derivative thereof, or a fusion of said fragment, variant or derivative thereof, which retains an antimicrobial activity of any one of SEQ ID NOs: 1 to 5.

It will be appreciated by persons skilled in the art that the term “amino acid”, as used herein, includes the standard twenty genetically-encoded amino acids and their corresponding stereoisomers in the ‘D’ form (as compared to the natural ‘L’ form), omega-amino acids other naturally-occurring amino acids, unconventional amino acids (e.g., a,a-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the collagen VI or polypeptide derived from collagen type VI of the composition of the first aspect comprise or consist of L-amino acids.

Where the polypeptide comprises an amino acid sequence according to a reference sequence (for example, SEQ ID NOs: 1 to 23), it may comprise additional amino acids at its N- and/or C-terminus beyond those of the reference sequence, for example, the polypeptide may comprise additional amino acids at its N-terminus. Likewise, where the polypeptide comprises a fragment, variant or derivative of an amino acid sequence according to a reference sequence, it may comprise additional amino acids at its N- and/or C-terminus.

In addition, where the composition comprises collagen type VI, one or more of the collagen type VI α chains may comprise additional amino acids at its N- and/or C-terminus, for example, the polypeptide may comprise additional amino acids at its N-terminus.

In one embodiment, the polypeptide of the composition comprises or consists of a fragment of the amino acid sequence according to a reference sequence (for example, a fragment of any one of SEQ ID NOs: 1 to 23). Thus, the polypeptide may comprise or consist of at least 5 contiguous amino acid of the reference sequence, for example at least 6, 7, 8, 9, 10, 11, 12, 13, 14, 15, 16, 17, 18, 19, 20, 21, 22, 23, 24, 25, 26 or 27 contiguous amino acids, e.g. of any one of SEQ ID NOs: 1 to 23.

It will be further appreciated by persons skilled in the art that the collagen VI or polypeptide of the composition of the first aspect may comprise or consist of a variant of the amino acid sequence according to a reference sequence (for example, a variant of any one of SEQ ID NOs: 1 to 23), or fragment of said variant. Such a variant may be non-naturally occurring.

By “variants” of the collagen VI or polypeptide we include insertions, deletions and substitutions, either conservative or non-conservative. For example, conservative substitution refers to the substitution of an amino acid within the same general class (e.g. an acidic amino acid, a basic amino acid, a non-polar amino acid, a polar amino acid or an aromatic amino acid) by another amino acid within the same class. Thus, the meaning of a conservative amino acid substitution and non-conservative amino acid substitution is well known in the art. In particular, we include variants of the polypeptide which exhibit an antimicrobial activity.

In one embodiment, the variant has an amino acid sequence which has at least 50% identity with the amino acid sequence according to a reference sequence (for example, SEQ ID NOs: 1 to 23) or a fragment thereof, for example at least 55%, 60%, 65%, 70%, 75%, 80%, 85%, 90%, 95%, 96%, 97%, 98% or at least 99% identity.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680, which is incorporated herein by reference).

The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

For example, in one embodiment, amino acids from the above reference sequences may be mutated in order to reduce proteolytic degradation of the polypeptide, for example by I,F to W modifications (see Strömstedt et al, Antimicrobial Agents Chemother 2009, 53, 593, which is incorporated herein by reference).

Variants may be made using the methods of protein engineering and site-directed mutagenesis well known in the art using the recombinant polynucleotides (see example, see Molecular Cloning: a Laboratory Manual, 3rd edition, Sambrook & Russell, 2000, Cold Spring Harbor Laboratory Press, which is incorporated herein by reference).

In a further embodiment, the polypeptide of the composition comprises or consists of an amino acid which is a species homologue of any one of the above amino acid sequences (e.g. SEQ ID NOS: 1 to 23). By “species homologue” we include that the polypeptide corresponds to the same amino acid sequence within an equivalent protein from a non-human species, i.e. which polypeptide exhibits the maximum sequence identity with of any one of SEQ ID NOS: 1 to 23 (for example, as measured by a GAP or BLAST sequence comparison). Typically, the species homologue polypeptide will be the same length as the human reference sequence (i.e. SEQ ID NOS: 1 to 23).

In a still further embodiment, the collagen VI or polypeptide of the composition comprises or consists of a fusion protein.

By “fusion” of a polypeptide we include an amino acid sequence corresponding to a reference sequence (for example, any one of SEQ ID NOs: 1 to 23, or a fragment or variant thereof) fused to any other polypeptide. For example, the said collagen VI or polypeptide may be fused to a polypeptide such as glutathione-S-transferase (GST) or protein A in order to facilitate purification of said polypeptide. Examples of such fusions are well known to those skilled in the art. Similarly, the said collagen VI or polypeptide may be fused to an oligo-histidine tag such as His6 or to an epitope recognised by an antibody such as the well-known Myc tag epitope. In addition, fusions comprising a hydrophobic oligopeptide end-tag may be used. Fusions to any variant or derivative of said collagen VI or polypeptide are also included in the scope of the invention. It will be appreciated that fusions (or variants or derivatives thereof) which retain desirable properties, such as an antimicrobial activity, are preferred.

The fusion may comprise a further portion which confers a desirable feature on the said collagen VI or polypeptide of the invention; for example, the portion may be useful in detecting or isolating the polypeptide, or promoting cellular uptake of the polypeptide. The portion may be, for example, a biotin moiety, a streptavidin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by persons skilled in the art that the collagen VI or polypeptide of the composition may comprise one or more amino acids that are modified or derivatised, for example by PEGylation, amidation, esterification, acylation, acetylation and/or alkylation.

As appreciated in the art, pegylated proteins may exhibit a decreased renal clearance and proteolysis, reduced toxicity, reduced immunogenicity and an increased solubility [Veronese, F. M. and J. M. Harris, Adv Drug Deliv Rev, 2002. 54(4): p. 453-6., Chapman, A. P., Adv Drug Deliv Rev, 2002. 54(4): p. 531-45] (incorporated herein by reference). Pegylation has been employed for several protein-based drugs including the first pegylated molecules asparaginase and adenosine deaminase [Veronese, F. M. and J. M. Harris, Adv Drug Deliv Rev, 2002. 54(4): p. 453-6., Veronese, F. M. and G. Pasut, Drug Discov Today, 2005. 10(21): p. 1451-8] (incorporated herein by reference).

In order to obtain a successfully pegylated protein, with a maximally increased half-life and retained biological activity, several parameters that may affect the outcome are of importance and should be taken into consideration. The PEG molecules may differ, and PEG variants that have been used for pegylation of proteins include PEG and monomethoxy-PEG. In addition, they can be either linear or branched [Wang, Y. S., et al., Adv Drug Deliv Rev, 2002. 54(4): p. 547-70] (incorporated herein by reference). The size of the PEG molecules used may vary and PEG moieties ranging in size between 1 and 40 kDa have been linked to proteins [Wang, Y. S., et al., Adv Drug Deliv Rev, 2002. 54(4): p. 547-70., Sato, H., Adv Drug Deliv Rev, 2002. 54(4): p. 487-504, Bowen, S., et al., Exp Hematol, 1999. 27(3): p. 425-32, Chapman, A. P., et al., Nat Biotechnol, 1999. 17(8): p. 780-3] (incorporated herein by reference). In addition, the number of PEG moieties attached to the protein may vary, and examples of between one and six PEG units being attached to proteins have been reported [Wang, Y. S., et al., Adv Drug Deliv Rev, 2002. 54(4): p. 547-70., Bowen, S., et al., Exp Hematol, 1999. 27(3): p. 425-32] (incorporated herein by reference). Furthermore, the presence or absence of a linker between PEG as well as various reactive groups for conjugation have been utilised. Thus, PEG may be linked to N-terminal amino groups, or to amino acid residues with reactive amino or hydroxyl groups (Lys, His, Ser, Thr and Tyr) directly or by using γ-amino butyric acid as a linker. In addition, PEG may be coupled to carboxyl (Asp, Glu, C-terminal) or sulfhydryl (Cys) groups. Finally, Gln residues may be specifically pegylated using the enzyme transglutaminase and alkylamine derivatives of PEG has been described [Sato, H., Adv Drug Deliv Rev, 2002. 54(4): p. 487-504] (incorporated herein by reference).

It has been shown that increasing the extent of pegylation results in an increased in vivo half-life. However, it will be appreciated by persons skilled in the art that the pegylation process will need to be optimised for a particular protein on an individual basis.

PEG may be coupled at naturally occurring disulphide bonds as described in WO 2005/007197, which is incorporated herein by reference. Disulfide bonds can be stabilised through the addition of a chemical bridge which does not compromise the tertiary structure of the protein. This allows the conjugating thiol selectivity of the two sulphurs comprising a disulfide bond to be utilised to create a bridge for the site-specific attachment of PEG. Thereby, the need to engineer residues into a peptide for attachment of to target molecules is circumvented.

A variety of alternative block copolymers may also be covalently conjugated as described in WO 2003/059973, which is incorporated herein by reference. Therapeutic polymeric conjugates can exhibit improved thermal properties, crystallisation, adhesion, swelling, coating, pH dependent conformation and biodistribution. Furthermore, they can achieve prolonged circulation, release of the bioactive in the proteolytic and acidic environment of the secondary lysosome after cellular uptake of the conjugate by pinocytosis and more favourable physicochemical properties due to the characteristics of large molecules (e.g. increased drug solubility in biological fluids). Block copolymers, comprising hydrophilic and hydrophobic blocks, form polymeric micelles in solution. Upon micelle disassociation, the individual block copolymer molecules are safely excreted.

Chemical derivatives of one or more amino acids may also be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by “collagen VI or polypeptide” we include peptidomimetic compounds which have an antimicrobial activity. The term “peptidomimetic” refers to a compound that mimics the conformation and desirable features of a particular peptide as a therapeutic agent.

For example, the collagen VI or polypeptides of the invention include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, which is incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the collagen VI or polypeptide of the invention may be a peptidomimetic compound wherein one or more of the amino acid residues are linked by a -γ(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will be appreciated that the collagen VI or polypeptide may conveniently be blocked at its N- or C-terminal region so as to help reduce susceptibility to exoproteolytic digestion.

A variety of uncoded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases.

Thus, exemplary collagen VI or polypeptides of the composition of the first aspect comprise terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminal region cysteines may circumvent problems of specificity and half-life sometime observed with linear peptides, by decreasing proteolysis and also increasing the rigidity of the structure, which may yield higher specificity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. Thus, the peptides of the composition of the present invention can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872, which is incorporated herein by reference. Other examples of cyclisation methods include cyclization through click chemistry, epoxides, aldehyde-amine reactions, as well as and the methods disclosed in U.S. Pat. No. 6,008,058, which is incorporated herein by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986), which is incorporated herein by reference, has been to design peptide mimics through the application of the transition state analogue concept in enzyme inhibitor design. For example, it is known that the secondary alcohol of staline mimics the tetrahedral transition state of the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification because of the stable structures formed by cyclisation and in view of the biological activities observed for cyclic peptides.

Thus, in one embodiment the collagen VI or polypeptide of the composition of the first aspect of the invention is linear. However, in an alternative embodiment, the polypeptide is cyclic.

It will be appreciated by persons skilled in the art that the polypeptides of the composition of the invention may be of various lengths. Typically, however, the polypeptide is between 10 and 200 amino acids in length, for example between 10 and 150, 15 and 100, 15 and 50, 20 and 40 or 25 and 35 amino acids in length. For example, the polypeptide may be at least 20 amino acids in length.

In one embodiment, the collagen VI or polypeptide of the composition is or comprises a recombinant polypeptide. Suitable methods for the production of such recombinant polypeptides are well known in the art, such as expression in prokaryotic or eukaryotic hosts cells (for example, see Sambrook & Russell, 2000, Molecular Cloning, A Laboratory Manual, Third Edition, Cold Spring Harbor, N.Y., the relevant disclosures in which document are hereby incorporated by reference).

Collagen VI or polypeptides of the composition of the invention can also be produced using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

It will be appreciated by persons skilled in the art that collagen VI or polypeptides of the composition of the invention may alternatively be synthesized artificially, for example using well known liquid-phase or solid phase synthesis techniques (such as t-Boc or Fmoc solid-phase peptide synthesis). For example, the polypeptides may be synthesized as described in Solid-Phase Peptide Synthesis (1997) Fields, Abelson & Simon (Eds), Academic Press (ISBN: 0-12-182190-0), which is incorporated herein by reference.

In some embodiments, the composition may comprise or consist of more than one different collagen type VI and/or polypeptide as defined in the first aspect. For example, the composition may comprise collagen type VI and further comprise a polypeptide comprising a collagen VI α3 chain. In another example, the composition may comprise a collagen VI α3 chain and one or more of the peptides GVR28 (SEQ ID NO: 1), FLY25 (SEQ ID NO: 2), FFL25 (SEQ ID NO: 3), VTT30 (SEQ ID NO: 4) and SFV33 (SEQ ID NO: 5).

As used herein, “polylysine” includes any compound that is a polymer of lysine monomer units, preferably joined by peptide bonds. For example, polylysine may include any compound comprising or consisting of 3 or more lysine residues that have been joined together by polymerisation at either the ε or a carbon position.

By “polymer” we mean a substance that is made up of multiple monomer units joined together by chemical bonds. Polymers can either form linear chains of molecules or three-dimensional networks depending on the type of molecule to be polymerised and the position of polymerisation.

By “polymerisation” we refer to the process used to form a polymer in which multiple monomer units form chemical bonds resulting in the formation of linear polymer chains or a three-dimensional network of molecules. In the case of polymerisation of amino acids such as lysine as described herein, polymerisation involves the formation of peptide bonds between amino acid monomers by reaction of an amino group and a carboxylic acid group. Formation of peptide bonds is a process that is well known in the art.

Polylysine may differ in both the enantiomer of lysine used (i.e. L or D lysine) and the carbon position of polymerisation (i.e. α or ε).

Lysine is found as two different enantiomers that differ in the arrangement of R-groups around the chiral centre, termed the L- and D-forms. Other forms of naming enantiomers are used in the art (for example R and S notation, where S-lysine corresponds to L-lysine and R-lysine corresponds to D-lysine), however L/D notation remains the most commonly used in relation to amino acids. The difference between L- and D-amino acids are well known in the art.

The polylysine of the invention may include polymers comprising or consisting of both L-lysine and D-lysine monomer units, or may comprise or consist of only L-lysine or only D-lysine monomer units in which case the polymers are termed poly-L-lysine (PLL) and poly-D-lysine (PDL), respectively.

In one embodiment the polylysine comprises or consists of poly-L-lysine (PDL) and/or poly-D-lysine (PLL).

Optical isomers such as PLL/PDL have different effects on plane-polarised light (light that travels in a single plane). One isomer will rotate the plane of this plane-polarised light clockwise, and the other will rotate it anticlockwise. This is how the isomers can be distinguished from one another.

In a further embodiment the polylysine comprises or consists of poly-L-lysine, optionally wherein 100% of the monomer units making up the polylysine are L-lysine.

In another embodiment, the polylysine comprises or consists of poly-D-lysine, optionally wherein 100% of the monomer units making up the polylysine are D-lysine.

The interchangeability of PLL and PDL is well known in the art as they both have the same charge-related properties. See, for example, Banker, G. and Goslin, K., Culturing Nerve Cells, MIT Press, Cambridge, p. 65 (1991).

In one embodiment, the polylysine may comprise a mixture of L-lysine and D-lysine monomer units. For example, 50% of the lysine monomers may be L-lysine and 50% may be D-lysine, or alternative ratios of D-lysine:L-lysine may be used, for example: 90:10; 80:20; 70:30; 60:40; 50:50; 40:60; 30:70; 20:80; 10:90.

As the R-group of lysine also contains an amino group (CH₂CH₂CH₂CH₂NH₂), there are two possible polymerization positions, either involving the a carbon or the ε carbon. By “α carbon” we mean the carbon atom in the backbone of the amino acid to which both the amine and carboxylic acid groups are attached. The carbon atoms in the lysine R group are then labeled sequentially (i.e. the first carbon of the R group attached to the a carbon is termed the β carbon, followed by the γ carbon, δ carbon and ε carbon atoms respectively in the carbon chain). Therefore, by “ε carbon” we mean the terminal carbon of the lysine R group to which the amine group is attached.

In one embodiment, the polylysine of the composition of the invention is polymerized at the ε carbon position. In an alternative embodiment, the polylysine is polymerized at the a carbon position.

In one embodiment, 100% of the polymerisation occurs at the ε carbon position. In an alternative embodiment, the polylysine comprises lysine monomer units polymerized at both the α and ε positions within the same molecule, for example 50% of the polymerisation may occur at the α position and 50% of the polymerization may occur at the ε position of the lysine monomer units. Polymerisation at the α or ε positions may also occur in different ratios, for example in the following ratios of α:ε polymerisation positions: 100:0; 90:10; 80:20; 70:30; 60:40; 50:50; 40:60; 30:70; 20:80; 10:90; 0:100.

The polylysine of the composition may be a mixture of different types of polylysine. In certain embodiments, the composition may comprise combinations of any one of the following: ε poly-L-lysine; α poly-L-lysine; ε poly-D-lysine; α poly-D-lysine. The different forms of polylysine may be found in different proportions within the composition, which may be adjusted to achieve optimal binding of the polypeptide to the surface in question. For example, in certain embodiments, the composition may comprise 50% ε poly-L-lysine and 50% ε poly-D-lysine. The composition may comprise equal or unequal amounts of all four variants of polylysine.

In one embodiment, the polylysine is poly-L-lysine (PLL) and is polymerised at the ε position of the lysine monomers.

In certain embodiments the polylysine may be modified. For example, either the lysine monomers that make up the polylysine may be modified prior to polymerisation or the polylysine itself may be modified after polymerisation.

Polylysine molecules are polymers which can vary in their molecular weight. Commercially available forms of polylysine are often found as compositions comprising polymers of a range of molecular weights rather than as a single molecular weight. Typically, this range of molecular weights of the polymers is from 30,000 Da to 300,000 Da.

In certain embodiments, the polylysine of the composition of the invention has a molecular weight in the range 30,000 Da to 300,000 Da. In other embodiments, the polylysine has a molecular weight in the range 50,000-250,000 Da; 70,000-200,000 Da; or 100,000-150,000 Da. In one embodiment, the polylysine molecules have a molecular weight in the range 30,000 to 70,000 Da. In one embodiment, the polylysine is poly-L-lysine and the poly-L-lysine molecules have a molecular weight in the range 30,000 to 70,000 Da.

Polylysine can also be categorized in terms of the number of lysine monomer units polymerised together. As the molecular weight of lysine is approximately 146 Da, polymers of polylysine in the molecular weight range 30,000 Da to 300,000 Da are made up of between approximately 200 and 2054 lysine monomer units. Therefore in some embodiments, the polylysine of a composition described herein is made up of between 200 and 2054 lysine monomer units. In other embodiments, the polylysine is poly-L-lysine and the molecules are made up of between 200 and 2054 L-lysine monomer units.

It would be clear to the skilled person that varying the range of molecular weight of the polylysine molecules in the composition would alter the number of lysine residues that make up each polymer molecule. Therefore, in other embodiments, the polylysine molecules of a composition described herein are made up of between: 342-1712 lysine monomers; 479-1369 lysine monomer units; 684-1027 lysine monomer units. In one embodiment, the polylysine molecules of a composition described herein are made up of between 200 and 480 lysine monomer units, corresponding to a molecular weight range of 30,000 to 70,000 Da. In other embodiments, the polylysine is poly-L-lysine and the molecules are made up of between 200 and 480 lysine monomer units.

In a further embodiment, the composition may additionally comprise a scaffold material, such as a biological and/or biodegradable material. The scaffold material may comprise or consist of collagen, for example collagen I.

It will be appreciated that the compositions described herein may be formulated for use in clinical medicine and/or veterinary medicine.

Thus, a second aspect of the invention provides a pharmaceutical composition comprising a composition according to the first aspect of the invention together with a pharmaceutically acceptable excipient, diluent, carrier, buffer or adjuvant.

As used herein, “pharmaceutical composition” means a therapeutically effective formulation for use in the treatment or prevention of disorders and conditions associated with microorganisms and microbial infections.

Additional compounds may also be included in the pharmaceutical compositions, such as other peptides, low molecular weight immunomodulating agents, receptor agonists and antagonists, and antimicrobial agents. Other examples include chelating agents such as EDTA, citrate, EGTA or glutathione.

The pharmaceutical compositions may be prepared in a manner known in the art that is sufficiently storage stable and suitable for administration to humans and animals. The pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the biological activity of the active ingredients, i.e. the antimicrobial polypeptide(s) of the composition. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide of the composition. The adjuvant may be one or more of colloidal silver, or zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as PHMB, cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, sucrose, mannitol, and cyclodextrines, which are added to the composition, e.g., for facilitating lyophilisation. Examples of polymers are starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, poly(lactic acid), poly(glycholic acid) or copolymers thereof with various composition, and polyvinylpyrrolidone, all of different molecular weight, which are added to the composition, e.g. for viscosity control, for achieving bioadhesion, or for protecting the active ingredient (applies to A-C as well) from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The pharmaceutical composition may also contain one or more mono- or di-saccharides such as xylitol, sorbitol, mannitol, lactitiol, isomalt, maltitol or xylosides, and/or monoacylglycerols, such as monolaurin. The characteristics of the carrier are dependent on the route of administration. One route of administration is topical administration. For example, for topical administrations, a preferred carrier is an emulsified cream comprising the active peptide, but other common carriers such as certain petrolatum/mineral-based and vegetable-based ointments can be used, as well as polymer gels, liquid crystalline phases and microemulsions.

It will be appreciated that the pharmaceutical compositions may comprise one or more of the collagen VI or polypeptides of the composition of the first aspect, for example one, two, three or four different peptides. By using a combination of different peptides the antimicrobial effect may be increased.

As discussed above, the collagen VI or polypeptide may be provided as a salt, for example an acid adduct with inorganic acids, such as hydrochloric acid, sulfuric acid, nitric acid, hydrobromic acid, phosphoric acid, perchloric acid, thiocyanic acid, boric acid etc. or with organic acid such as formic acid, acetic acid, haloacetic acid, propionic acid, glycolic acid, citric acid, tartaric acid, succinic acid, gluconic acid, lactic acid, malonic acid, fumaric acid, anthranilic acid, benzoic acid, cinnamic acid, p-toluenesulfonic acid, naphthalenesulfonic acid, sulfanilic acid etc. Inorganic salts such as monovalent sodium, potassium or divalent zinc, magnesium, copper calcium, all with a corresponding anion, may be added to improve the biological activity of the antimicrobial composition.

The pharmaceutical compositions of the invention may also be in the form of a liposome, in which the polypeptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, which is incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microshperes. Preparations of such microspheres can be found in U.S. Pat. No. 5,851,451 and in EP 213 303, which are incorporated herein by reference.

The pharmaceutical compositions of the invention may also be formulated with micellar systems formed by surfactants and block copolymers, preferably those containing poly(ethylene oxide) moieties for prolonging bloodstream circulation time.

The pharmaceutical compositions of the invention may also be in the form of polymer gels, where polymers such as starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, chitosan, carageenans, hyaluronic acid and derivatives thereof, polyacrylic acid, polyvinyl imidazole, polysulphonate, polyethylenglycol/polyethylene oxide, polyethylene-oxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

Alternatively, the collagen VI or polypeptide of the composition may be dissolved in saline, water, polyethylene glycol, propylene glycol, ethanol or oils (such as safflower oil, corn oil, peanut oil, cottonseed oil or sesame oil), tragacanth gum, and/or various buffers.

The pharmaceutical composition may also include ions and a defined pH for potentiation of action of anti-microbial polypeptides.

The above compositions of the invention may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

It will be appreciated by persons skilled in the art that the pharmaceutical compositions of the invention may be administered locally or systemically. Routes of administration include topical (e.g. ophthalmic), ocular, nasal, pulmonary, buccal, parenteral (intravenous, subcutaneous, and intramuscular), oral, vaginal and rectal. Also administration from implants is possible. Suitable preparation forms are, for example granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterised by long-range order but short-range disorder (examples include lamellar, hexagonal and cubic phases, either water- or oil continuous), or their dispersed counterparts, gels, ointments, dispersions, suspensions, creams, aerosols, droplets or injectable solution in ampoule form and also preparations with protracted release of active compounds, in whose preparation excipients, diluents, adjuvants or carriers are customarily used as described above. The pharmaceutical composition may also be provided in bandages, plasters or in sutures or the like.

In a particular embodiment, the pharmaceutical composition is suitable for oral administration, parenteral administration or topical administration. For example, the pharmaceutical composition may be suitable for topical administration (e.g. ophthalmic administration, in the form of a spray, lotion, paste or drops etc.).

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the, activity of the compound, manner of administration, nature and severity of the disorder, age and body weight of the patient different doses may be needed. The administration of the dose can be carried out both by single administration in the form of an individual dose unit or else several smaller dose units and also by multiple administrations of subdivided doses at specific intervals.

The pharmaceutical compositions of the invention may be administered alone or in combination with other therapeutic agents, such as additional antibiotic, anti-inflammatory, immunosuppressive, vasoactive and/or antiseptic agents (such as anti-bacterial agents, anti-fungicides, anti-viral agents, and anti-parasitic agents). Examples of suitable additional antibiotic agents include penicillins, cephalosporins, carbacephems, cephamycins, carbapenems, monobactams, aminoglycosides, glycopeptides, quinolones, tetracyclines, macrolides, and fluoroquinolones. Antiseptic agents include iodine, silver, copper, clorhexidine, polyhexanide and other biguanides, chitosan, acetic acid, and hydrogen peroxide. Likewise, the pharmaceutical compositions may also contain anti-inflammatory drugs, such as steroids and macrolactam derivatives.

Such additional therapeutic agents may be incorporated as part of the same pharmaceutical composition or may be administered separately.

It will be appreciated by persons skilled in the art that the compositions of the invention, or pharmaceutical compositions thereof, may be applied to medical devices and other products the implantation into or application of which to the human or animal body is associated with the risk of infection by a microbial agent.

Thus, a third aspect of the invention provides a medical device, implant, wound care product, or material for use in the same, which is coated, impregnated, admixed or otherwise associated with a composition according to the first aspect of the invention or a pharmaceutical composition according to the second aspect of the invention.

Such a medical device, implant, wound care product, or material for use in the same may come into contact with the human body or component thereof (e.g. blood).

In one embodiment, the medical device, implant, wound care product, or material for use in the same is for use in by-pass surgery, extracorporeal circulation, wound care and/or dialysis.

The composition may be coated, painted, sprayed or otherwise applied to or admixed with a suture, prosthesis, implant, wound dressing, catheter, lens, skin graft, skin substitute, fibrin glue or bandage, etc. In so doing, the composition may impart improved antimicrobial or wound healing properties to the device or material.

By “implant”, we include:
(a) A catheter (for example, for intravascular or urinary tract use);
(b) A stent (for example, a coronary stent);
(c) A shunt (for example, a cerebrospinal shunt);
(d) An intubating or tracheotomy tube;
(e) An ophthalmic device (for example, contact lenses, scleral buckles and intraocular lenses);
(f) A joint prosthesis (i.e. arthroplasty and implantation of other orthopaedic devices);
(g) An artificial heart valve;
(h) A breast implant;
(i) An implantable drug delivery device (for example, active pumps and passive solid implants).

In some preferred embodiments, the implant is a prosthesis or other orthopaedic device. In some preferred embodiments, the prosthesis is a knee joint prosthesis. In other preferred embodiments, the prosthesis is a hip joint prosthesis. Further examples of such implants are well known in the art. In further preferred embodiments the implant is a prosthesis or other orthopaedic device comprising or consisting of titanium or a titanium alloy. In one embodiment, the prosthesis or other orthopaedic device comprises or consists of titanium or a titanium alloy coated with a composition or pharmaceutical composition as defined herein.

In other embodiments, the implant is selected from: bone replacement devices, bone fixation devices, bone plates, stems of artificial hip joints, artificial organs, artificial intervertebral discs, spinal rods, maxillofacial plates, stent grafts, percutaneous devices and pacemakers. In one embodiment these implants comprise or consist of titanium or a titanium alloy. In further embodiments, the implant comprises or consists of titanium or a titanium alloy coated with a composition or pharmaceutical composition as defined herein.

In one embodiment, the device or material is coated with the composition or pharmaceutical composition of the invention (or the polypeptide component thereof). By ‘coated’ we mean that the composition or pharmaceutical composition is applied to the surface of the device or material. Thus, the device or material may be painted or sprayed with a solution comprising a composition or pharmaceutical composition of the invention (or polypeptide thereof). Alternatively, the device or material may be dipped in a reservoir of a solution comprising a composition or pharmaceutical composition of the invention.

In a one embodiment, the medical device, implant, wound care product, or material for use in the same is coated with a composition or pharmaceutical composition as defined herein.

In an alternative embodiment, the device or material is impregnated with a pharmaceutical composition of the invention (or collagen VI or polypeptide thereof). By ‘impregnated’ we mean that the pharmaceutical composition is incorporated or otherwise mixed with the device or material such that it is distributed throughout.

For example, the device or material may be incubated overnight at 4° C. in a solution comprising a composition or pharmaceutical composition of the invention. Alternatively, a composition or pharmaceutical composition of the invention may be immobilised on the device or material surface by evaporation or by incubation at room temperature.

In a further alternative embodiment, a polypeptide of the composition of the invention is covalently linked to the device or material, e.g. at the external surface of the device or material. Thus, a covalent bond is formed between an appropriate functional group on the polypeptide and a functional group on the device or material. For example, methods for covalent bonding of polypeptides to polymer supports include covalent linking via a diazonium intermediate, by formation of peptide links, by alkylation of phenolic, amine and sulphydryl groups on the binding protein, by using a poly functional intermediate e.g. glutardialdehyde, and other miscellaneous methods e.g. using silylated glass or quartz where the reaction of di- and trialkoxysilanes permits derivatisation of the glass surface with many different functional groups. For details, see Enzyme immobilisation by Griffin, M., Hammonds, E. J. and Leach, C. K. (1993) In Technological Applications of Biocatalysts (BIOTOL SERIES), pp. 75-118, Butterworth-Heinemann, incorporated herein by reference. See also the review article entitled Biomaterials in Tissue Engineering' by Hubbell, J. A. (1995) Science 13:565-576, which is incorporated herein by reference.

In one embodiment, the medical device, implant, wound care product, or material comprises or consists of a polymer. Suitable polymers may be selected from the group consisting of polyesters (e.g. polylactic acid, polyglycolic acid or poly lactic acid-glycolic acid copolymers of various composition), polyorthoesters, polyacetals, polyureas, polycarbonates, polyurethanes, polyamides) and polysaccharide materials (e.g. cross-linked alginates, hyaluronic acid, carageenans, gelatines, starch, cellulose derivatives).

Alternatively, or in addition, the medical device, implant, wound care product, or material may comprise or consists of metals (e.g. titanium, stainless steel, gold, titanium), metal oxides (silicon oxide, titanium oxide) and/or ceramics (apatite, hydroxyapatite).

In one embodiment, the medical device, implant, wound care product or material for use in the same comprises or consists of titanium.

By “comprises or consists of titanium” we include materials made solely of pure titanium and also materials that comprise titanium in combination with other elements. For example, by “comprises or consists of titanium” we also include materials that are or comprise titanium alloys (for example alloys of titanium with nickel, vanadium and/or aluminium). We also include medical devices, implants, wound care products and materials for use in the same that have at least one component comprising or consisting of titanium and/or one or more titanium alloys. We also include medical devices, implants, wound care products and materials for use in the same that have a coating comprising or consisting of titanium, for example a coating comprising or consisting of titanium or titanium nitride. It will be clear therefore to the skilled person that mention herein of titanium is also meant to include reference to titanium alloy.

Thus, in one embodiment the titanium is commercially pure titanium (CP Ti). Alternatively, in specific embodiments the titanium is an alloy, for example a Ti₆Al₄V alloy or a nickel-titanium alloy (Nitinol).

Such materials may be in the form of macroscopic solids/monoliths, as chemically or physicochemically cross-linked gels, as porous materials, or as particles.

Medical devices, implants, wound care products, and materials of the invention may be made using methods well known in the art.

In certain embodiments, the composition of the first aspect of the invention is coated onto a biological scaffold, such as a collagen scaffold, for example a collagen I scaffold. In one embodiment the collagen scaffold may be used directly as a medical device, implant or wound care product or material for use in the same. In other embodiments, the collagen scaffold is a component of a medical device, implant, wound care product or material for use in the same. For example, the collagen scaffold may be coated onto the surface of such a medical device, implant, wound care product or material for use in the same.

In certain embodiments, the scaffold is a collagen scaffold, such as a collagen I scaffold. In certain embodiments, the polylysine forms an intermediate layer between the scaffold and the collagen VI polypeptide.

In other embodiments, the composition of the first aspect of the invention is coated directly onto the surface of a medical device, implant, wound care product or material for use in the same, wherein the medical device, implant, wound care product or material for use in the same comprises or consists of titanium or a titanium alloy. In some embodiments the coated titanium surface may be used directly as a medical device, implant or wound care product or material for use in the same. In other embodiments, the coated titanium surface is a component of a medical device, implant, wound care product or material for use in the same.

In certain embodiments, the polylysine forms an intermediate layer between the titanium surface and the collagen VI polypeptide.

In some embodiments the coated titanium surface may be used directly as an implant comprising or consisting of titanium or a titanium alloy, for example as a joint prosthesis or other orthopaedic device, for example as knee joint prostheses or hip joint prostheses. In further embodiments, the coated titanium surface may be used directly as an implant comprising or consisting of titanium or a titanium alloy, for example as bone replacement devices, bone fixation devices, bone plates, stems of artificial hip joints, artificial organs, artificial intervertebral discs, spinal rods, maxillofacial plates, stent grafts, percutaneous devices and pacemakers.

By “titanium surface” we include all medical devices, implants, wound care products or materials for use in the same with surfaces that comprise or consist of titanium or a titanium alloy.

It will be appreciated that any of the medical devices, implants, wound care products, and materials of the invention may be used for any of the medical uses disclosed herein.

In one embodiment the medical device, implant, wound care product or material for use in the same is coated with a composition as defined in the first aspect, such that the composition or pharmaceutical composition is applied to the surface of said material, resulting in the binding of the collagen VI or polypeptide of the composition to the surface of the material.

The skilled person would be aware of techniques available in the art for determining the level of binding of a polypeptide component of a composition to the surface of a material, such as radioactive labelling of peptides. An example of this is labelling with a radioactive isotope of iodine (e.g. iodine 131) by labelling lysine or tyrosine residues, and measuring the level of radioactivity produced by a coated material, which is proportional to the level of binding achieved. The coating efficiencies of the different biomolecules can be assessed by determining the ratio between bound and free 131-iodine radioactivity associated with the material by determining radioactivity as cpm values in a γ-counter, i.e. at a measurement of 100% binding an undetectable amount of radioactive iodine 131 is not bound to the surface of the material.

Other techniques for measuring percentage binding of a peptide or protein to a surface include but are not limited to: spectroscopic assays; radioactivity based binding assays; ellipsometry; single-oscillation quartz crystal thin-film thickness monitoring; fluorescence based binding assays; surface plasmon resonance (SPR) and atomic force microscopy (AFM).

Compositions comprising the collagen VI polypeptide and polylysine as defined in the first aspect of the invention unexpectedly achieve surprisingly higher percentage binding than compositions not containing polylysine.

In one embodiment the percentage binding of collagen VI or the polypeptide of the composition disclosed herein to the medical device, implant, wound care product, or material for use in the same is at least 40%, 50%, 60%, 70%, 75%, 80%, 85%, 90%, 95%, or at least 95.3%. In one embodiment, the percentage binding is at least 50%. In a further embodiment, the percentage binding is at least 85%, or at least 86%, for example at least 86.8%.

As discussed herein, the compositions of the invention comprising polylysine achieve higher percentage binding to a surface than compositions not comprising polylysine.

In one embodiment the percentage binding of collagen VI or the polypeptide of the composition to the medical device, implant, wound care product, or material for use in the same is greater than the percentage binding achieved by a composition that does not contain polylysine, for example; at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or at least 249% greater. In one embodiment, the percentage binding achieved by the compositions containing polylysine is at least 50% greater, preferably at least 59% greater than compositions not containing polylysine. In one embodiment, the percentage binding achieved by compositions containing polylysine is at least 100% greater, preferably at least 109% greater than compositions not containing polylysine. In one embodiment, the percentage binding achieved by the compositions containing polylysine is at least 140% greater, preferably at least 141% greater than compositions not containing polylysine.

In some embodiments, the composition contains an appropriate amount of polylysine required to achieve improved binding of the polypeptide to the surface of a material as defined herein. In some embodiments, the concentration of polylysine in the composition is between 0.1 and 1 mg/ml, for example is between: 0.1-0.3 mg/ml; 0.15-0.25 mg/ml; 0.16-0.24 mg/ml; 0.17-0.23 mg/ml; 0.18-0.22 mg/ml; or 0.19-0.21 mg/ml. In one embodiment, the concentration of polylysine in the composition is in the range 0.15-0.25 mg/ml. In one embodiment, the concentration of polylysine in the composition is 0.2 mg/ml.

A fourth aspect of the invention provides a method of preparing a medical device, implant, wound care product or material for use in the same, comprising the step of coating, impregnating, admixing or otherwise associating the medical device, implant, wound care product, or material for use in the same with the composition or pharmaceutical composition described in the first or second aspects.

The skilled person would understand that the general methods of coating, impregnating and admixing as described above could also be applied to methods of preparing medical devices, implants, wound care products or materials for use in the same according to this aspect of the invention.

A fifth aspect of the invention provides a method of preparing a medical device, implant, wound care product or material for use in the same comprising the following steps:
(i) preparing a composition comprising collagen VI or a polypeptide as defined in the first aspect and polylysine as defined in the first aspect; and
(ii) coating, impregnating, admixing or otherwise associating the medical device, implant, wound care product, or material for use in the same with the composition prepared in step (i).

Thus, medical devices, implants, wound care products and materials for use herein may be coated, impregnated, admixed or otherwise associated with a composition of the first aspect of the invention by utilising a single composition comprising both the collagen VI or polypeptide of the first aspect and polylysine of the first aspect. The medical device, implant, wound care product or material for use in the same may preferably be coated with such a composition.

In one embodiment, the medical device, implant, wound care product or material for use in the same comprises or consists of a titanium surface.

A sixth aspect of the invention provides a method of preparing a medical device, implant, wound care product or material for use in the same comprising the following steps:
(i) coating, impregnating, admixing or otherwise associating the medical device, implant, wound care product, or material for use in the same with polylysine as defined in the first aspect; and
(ii) coating, impregnating, admixing or otherwise associating the medical device, implant, wound care product, or material for use in the same produced in step (i) with collagen VI or a polypeptide as defined in the first aspect.

Thus, medical devices, implants, wound care products and materials for use herein may be prepared by first coating, impregnating, admixing or otherwise associating the material with polylysine as defined in the first aspect, followed by an additional step in which the collagen VI or polypeptide as defined in the first aspect is applied subsequently.

In certain embodiments, in step (i) the polylysine in step is coated onto a scaffold, such as a biological scaffold (e.g. a collagen such as collagen I) prior to coating, impregnating, admixing or otherwise associating the material with a collagen VI or polypeptide as defined in the first aspect. The scaffold may be present on a titanium surface.

In other embodiments, the polylysine is not coated onto a scaffold prior to coating, impregnating, admixing or otherwise associating the material with a collagen VI or polypeptide as defined in the first aspect. For example, in some embodiments, the polylysine is coated directly onto a surface, for example a surface comprising or consisting of titanium or a titanium alloy, prior to coating, impregnating, admixing or otherwise associating the material with a collagen VI or polypeptide as defined in the first aspect.

In other embodiments, the polylysine can be mixed with a collagen VI or polypeptide as defined in the first aspect prior to coating, impregnating, admixing or otherwise associating the mixture with a collagen scaffold or other surface e.g. a surface comprising or consisting of titanium or a titanium alloy.

In some embodiments, the medical device, implant, wound care product, or material for use in the same comprises or consists of a titanium surface.

In certain embodiments, the method of preparing a medical device, implant, wound care product or material for use in the same, comprises one or more of the following steps:
(i) incubating a scaffold, e.g. a collagen scaffold, with a solution of polylysine;
(ii) washing the scaffold (e.g. in distilled water);
(iii) drying the scaffold (e.g. by air drying);
(iv) coating the scaffold with collagen VI or a collagen VI polypeptide as defined in the first aspect of the invention (e.g. by plunging the scaffold into a solution containing the collagen VI or collagen VI polypeptide);
(v) incubating the scaffold in the collagen VI solution (e.g. overnight)
(vi) drying the scaffold (e.g. by air drying).

In certain embodiments of the above method, the collagen scaffold is a collagen I scaffold. In one embodiment, the collagen scaffold is disc shaped.

In certain embodiments of the above method, the polylysine of step (i) is poly-L-lysine. In some embodiments, the concentration of the polylysine solution is approximately 0.2 mg/ml. In one embodiment, the polylysine solution of step (i) is an approximately 0.2 mg/ml solution of poly-L-lysine. In some embodiments, the collagen scaffolds are incubated in the solution of polylysine at approximately 60° C. In some embodiment, the collagen scaffolds are incubated in the solution of polylysine for approximately two hours.

In certain embodiments of the above method, the concentration of collagen VI in the collagen VI solution of step (iv) is approximately 150 mM. In some embodiments, the concentration of collagen VI peptides in the collagen VI solution of step (iv) is approximately 2-3 mM. In some preferred embodiments, the concentration of collagen VI peptides in the collagen VI solution of step (iv) is approximately 2-3 μM.

In certain embodiments, the collagen scaffolds are incubated in the collagen VI solution overnight, i.e. for approximately 16 hours. In some embodiments, the collagen scaffolds are incubated with the collagen VI solution at approximately 4° C.

In other embodiments of step (i) above the polylysine in step (i) is coated onto the surface of the medical device, implant, wound care product, or material for use in the same, prior to coating, impregnating, admixing or otherwise associating the material with a collagen VI or polypeptide as defined in the first aspect. In some embodiments, the medical device, implant, wound care product, or material for use in the same comprises or consists of a titanium surface.

In certain embodiments, the method of preparing a medical device, implant, wound care product or material for use in the same, comprises one or more of the following steps:
(i) incubating a medical device, implant, wound care product or material for use in the same, e.g. a titanium surface, with a solution of polylysine;
(ii) washing the surface (e.g. in distilled water);
(iii) drying the surface (e.g. by air drying);
(iv) coating the surface with collagen VI or a collagen VI polypeptide as defined in the first aspect of the invention (e.g. by plunging the surface into a solution containing the collagen VI or collagen VI polypeptide);
(v) incubating the surface in the collagen VI solution (e.g. overnight)
(vi) drying the surface (e.g. by air drying).

In certain embodiments of the above method, the polylysine of step (i) is poly-L-lysine. In some embodiments, the concentration of the polylysine solution is approximately 0.2 mg/ml. In one embodiment, the polylysine solution of step (i) is an approximately 0.2 mg/ml solution of poly-L-lysine. In some embodiments, the titanium surface is incubated in the solution of polylysine at approximately 60° C. In some embodiments, the titanium surfaces are incubated in the solution of polylysine for approximately two hours.

In certain embodiments of the above method, the concentration of collagen VI in the collagen VI solution of step (iv) is approximately 150 mM. In some embodiments, the concentration of collagen VI peptides in the collagen VI solution of step (iv) is approximately 2-3 mM. In some preferred embodiments, the concentration of collagen VI peptides in the collagen VI solution of step (iv) is approximately 2-3 μM.

A seventh aspect of the invention provides a kit comprising:
(i) a composition according to any the first aspect of the invention or a pharmaceutical composition according to the second aspect of the invention or a medical device, implant, wound care product, or material for use in the same according to a third aspect of the invention, and
(ii) instructions for use.

An eighth aspect of the invention provides a kit comprising:
(i) a collagen VI or polypeptide according to in the first aspect of the invention;
(ii) polylysine according to the first aspect of the invention;
(iii) instructions for use.

In certain embodiments of the kits of the seventh and eighth aspects, the kit additionally comprises a scaffold material, such as a biological and/or biodegradable scaffold. The scaffold may comprise or consist of collagen, e.g. collagen I.

In other embodiments of the kits of the seventh and eighth aspects, the kit additionally comprises a material comprising or consisting of titanium or a titanium alloy, for example a medical device, implant, wound care produced or material for use in the same comprising or consisting of titanium.

A ninth aspect of the invention provides a composition according to the first aspect of the invention, or a pharmaceutical composition according to the second aspect of the invention for use in medicine.

A tenth aspect of the invention provides a composition according to the first aspect of the invention, or a pharmaceutical composition according to the second aspect of the invention for use in the curative and/or prophylactic treatment of microbial infections.

The term ‘prophylactic’ is used to encompass the use of a composition or formulation described herein which either prevents or reduces the likelihood of a condition or disease state in a patient or subject.

By “microbial infections” we include infections caused by microorganisms as described above.

For example, in one embodiment the microbial infection to be treated is a bacterial infection.

The microbial infection to be treated may be an acute or a systemic infection.

In one embodiment, the microbial infection is resistant to one or more conventional antibiotic agents (as discussed above).

In one embodiment, the microbial infection to be treated is caused by a microorganism selected from the group consisting of: Pseudomonas aeruginosa, Staphylococcus aureus, Escherichia coli and Streptococcus pyogenes.

In a further embodiment, the microbial infection is caused by a microorganism selected from the group consisting of: multidrug-resistant Staphylococcus aureus (MRSA) (methicillin-resistant Staphylococcus aureus) and multidrug-resistant Pseudomonas aeruginosa (MRPA).

It will be appreciated by persons skilled in the art that the compositions of the invention may be co-administered in combination with one or more known or conventional agents for the treatment of the particular disease or condition. By ‘co-administer’ it is meant that the present compositions are administered to a patient such that the components of the composition as well as the co-administered compound may be found in the patient's body (e.g. in the bloodstream) at the same time, regardless of when the compounds are actually administered, including simultaneously.

Therefore, in one embodiment the composition or pharmaceutical composition is for use in combination with one or more additional antimicrobial agents, such as the conventional antibiotics described above. Alternatively, or in addition, the additional antimicrobial agents may be an antimicrobial polypeptide or protein, such as LL-37 and collagen type VI protein, or for example selected from group consisting of defensins, gramicidin S, magainin, cecropin, histatin, hyphancin, cinnamycin, burforin 1, parasin 1 and protamines, and fragments, variants and fusion thereof which retain, at least in part, the antimicrobial activity of the parent protein.

An eleventh aspect of the invention provides a composition according to the first aspect of the invention or a pharmaceutical composition according to the second aspect of the invention for use in wound care.

By “wound care” we include the treatment of wounds, promoting wound closure, preventing and/or treating wound infection and/or ulcers, wherein the wound may be extracorporeal or intracorporeal. Use in wound care therefore includes compositions comprising polypeptides which are able to aid (for example, accelerate) the wound healing process and/or to prevent infection of the wound. For example, the collagen VI or polypeptide of the composition may be used in a wound care product, such as a cream, gel, ointment, dressing or plaster, which is capable of enhancing epithelial regeneration and/or healing of wound epithelia and/or wound stroma. In one embodiment, the polypeptide is capable of enhancing the proliferation of epithelial and/or stromal cells through a non-lytic mechanism.

It will be appreciated that the collagen VI and polypeptides having wound healing properties may have a primary or ancillary role in the function of the wound care products of the invention.

In one embodiment, the collagen VI or polypeptide or pharmaceutical composition is administered in combination with an additional antimicrobial agent, as described above.

A twelfth aspect of the invention provides the use of a composition according to the first aspect of the invention, or a pharmaceutical composition according to the second aspect of the invention in the manufacture of a medicament for the treatment of microbial infections, as described above.

A thirteenth aspect of the invention provides the use of a composition according to the first aspect of the invention, or a pharmaceutical composition according to the second aspect of the invention in the manufacture of a medicament for the treatment of wounds, as described above.

A fourteenth aspect of the invention provides a method of treating an individual with a microbial infection, the method comprising the step of administering to an individual in need thereof an effective amount of a composition according to the first aspect of the invention, or a pharmaceutical composition according to the second aspect of the invention.

A fifteenth aspect of the invention provides a method of treating a wound in an individual, the method comprising the step of administering to an individual in need thereof an effective amount of a composition according to the first aspect of the invention, or a pharmaceutical composition according to the second aspect of the invention.

The term ‘effective amount’ is used herein to describe concentrations or amounts of compositions or pharmaceutical compositions according to the present invention which may be used to produce a favourable change in a disease or condition treated, whether that change is a remission, a favourable physiological result, a reversal or attenuation of a disease state or condition treated, the prevention or the reduction in the likelihood of a condition or disease state occurring, depending upon the disease or condition treated. Where compositions or pharmaceutical compositions of the invention are used in combination, each of the compositions or pharmaceutical compositions may be used in an effective amount, wherein an effective amount may include a synergistic amount.

It will be appreciated by persons skilled in the art that the compositions and pharmaceutical formulations of the present invention have utility in both the medical and veterinary fields. Thus, the methods of the invention may be used in the treatment of both human and non-human animals (such as horses, dogs and cats). Preferably, however, the patient is human.

For veterinary use, a composition of the invention is administered as a suitably acceptable formulation in accordance with normal veterinary practice and the veterinary surgeon will determine the dosing regimen and route of administration which will be most appropriate for a particular animal.

A sixteenth aspect of the invention provides a method for killing microorganisms in vitro comprising contacting the microorganisms with a composition according to the first aspect of the invention or a pharmaceutical composition according to the second aspect of the invention. For example, the composition or pharmaceutical composition may also be used in the form of a sterilising solution or wash to prevent the growth of microorganisms on a surface or substrate, such as in a clinical environment (e.g. surgical theatre) or a domestic environment (e.g. a kitchen work surface, washing clothes such as bed linen).

In one embodiment the antimicrobial compound may be in solution at a concentration of 1 to 100 μg/ml.

In one embodiment the solution further comprises a surface-active agent or surfactant. Suitable surfactants include anionic surfactants (e.g. an aliphatic sulphonate), amphoteric and/or zwitterionic surfactants (e.g. derivatives of aliphatic quaternary ammonium, phosphonium and sulfonium compounds) and nonionic surfactants (e.g. aliphatic alcohols, acids, amides or alkyl phenols with alkylene oxides)

Conveniently, the surface-active agent is present at a concentration of 0.5 to 5 weight percent.

The sterilising solutions are particularly suited for use in hospital environments. For example, the sterilising solutions may be used to sterilise surgical instruments and surgical theatre surfaces, as well as the hands and gloves of theatre personnel. In addition, the sterilising solutions may be used during surgery, for example to sterilise exposed bones. In all cases, the solution is applied to the surface to be sterilised.

The composition or pharmaceutical composition may also be used to disinfect blood and blood products and in the diagnosis of bacterial contamination or infection.

In both in vitro and in vivo uses, the pharmaceutical composition or polypeptide is preferably exposed to the target microorganisms (or surface/area to be treated) for at least five minutes. For example, the exposure time may be at least 10 minutes, 20 minutes, 30 minutes, 40 minutes, 50 minutes, 1 hour, 2 hours, 3, hours, 5 hours, 12 hours and 24 hours.

In one embodiment of the composition or pharmaceutical composition for use according to the ninth, tenth or eleventh aspects of the invention, the use according to the twelfth or thirteenth aspects of the invention, or the method according to the fourteenth, fifteenth or sixteenth aspects of the invention, the composition or pharmaceutical composition is coated or impregnated onto, or admixed or otherwise associated with, a medical device, implant, wound care product, or material for use in the same.

The listing or discussion of an apparently prior-published document in this specification should not necessarily be taken as an acknowledgement that the document is part of the state of the art or is common general knowledge.

Preferred, non-limiting examples which embody certain aspects of the invention will now be described, with reference to the following figures:

### DESCRIPTION OF THE FIGURES

FIG. 1: Quantitative evaluation of the coating efficiency of collagen I scaffolds with native collagen VI fibrils and other biomolecules. Binding of native collagen VI microfibrils to collagen I scaffolds with 1 mm (thin biodegradable scaffold), or 2 mm (thick biodegradable scaffold) thickness, respectively, was assessed by 131-iodine assays as indicated in the Figure. The coating efficiency with and without precoating with PLL was compared to the cathelicidin peptide LL-37, and the host defence peptides and proteins NAT26, HKH20, GGL27, α defensins 3-6 and β defensins 1-4. Coating with collagen VI microfibrils is considerably enhanced with PLL as an intermediate layer as compared to the controls.

FIG. 2: Quantitative evaluation of the coating efficiency of collagen I scaffolds with different collagen VI peptides. Binding of native collagen VI microfibrils to collagen I scaffolds with 1 mm (thin biodegradable scaffold), or 2 mm (thick biodegradable scaffold) thickness, respectively, was assessed by 131-iodine assays as indicated in the Figure. The coating efficiency with and without precoating with PLL was compared to the different collagen VI alpha-chains, and the bioactive collagen VI peptides GVR28, FYL25, FFL25, VTT30, and SFV33. Coating with the collagen VI alpha-3 chain and its derived peptides is considerably enhanced with PLL as an intermediate layer as compared to the other parts of the collagen VI molecule.

FIG. 3: Quantitative evaluation of the coating efficiency of titanium surfaces with native collagen VI fibrils and other biomolecules. Binding of native collagen VI microfibrils to titanium surfaces was assessed by 131-iodine assays as indicated in FIG. 3. The coating efficiency with and without precoating with PLL was compared to the cathelicidin peptide LL-37, and the host defence peptides and proteins NAT26, HKH20, GGL27, α defensins 3-6 and β defensins 1-4. Coating with collagen VI microfibrils is considerably enhanced with PLL as an intermediate layer compared to the controls.

FIG. 4: Quantitative evaluation of the coating efficiency of titanium surfaces with different collagen VI peptides. Binding of native collagen VI microfibrils to titanium surfaces was assessed by 131-iodine assays as indicated in FIG. 4. The coating efficiency with and without precoating with PLL was compared to the different collagen VI alpha-chains, and the bioactive collagen VI peptides GVR28, FYL25, FFL25, VTT30, and SFV33. Coating with the collagen VI alpha-3 chain and its derived peptides is considerably enhanced with PLL as an intermediate layer compared to the other parts of the collagen VI molecule.

FIG. 5: Quantitative evaluation of the Gram-negative bacterial killing efficiency on titanium surfaces coated with different collagen VI peptides. Killing of Pseudomonas aeruginosa bacteria on the different titanium surfaces was assessed by scanning electron microscopy assays. The killing efficiency with and without precoating with PLL was compared to the different collagen VI alpha-chains, and the bioactive collagen VI peptides GVR28, FYL25, FFL25, VTT30, and SFV33. Killing by the collagen VI alpha-3 chain and its derived peptide SFV33 is considerably enhanced with PLL as an intermediate layer as compared to the other parts of the collagen VI molecule.

FIG. 6: Quantitative evaluation of the Gram-positive bacterial killing efficiency on titanium surfaces coated with different collagen VI peptides. Killing of Staphylococcus aureus bacteria on the different titanium surfaces was assessed by scanning electron microscopy assays. The killing efficiency with and without precoating with PLL was compared to the different collagen VI alpha-chains, and the bioactive collagen VI peptides GVR28, FYL25, FFL25, VTT30, and SFV33. Killing by the collagen VI alpha-3 chain and its derived peptide SFV33 is considerably enhanced with PLL as an intermediate layer as compared to the other parts of the collagen VI molecule.

FIG. 7: Quantitative evaluation of the fibroblast adherence efficiency on titanium surfaces coated with different collagen VI peptides. Adherence of skin fibroblasts on the different titanium surfaces was assessed by scanning electron microscopy assays. The cell adherence efficiency with and without precoating with PLL was compared to the different collagen VI alpha-chains, and the bioactive collagen VI peptides GVR28, FYL25, FFL25, VTT30, and SFV33. Fibroblast adherence mediated by the collagen VI alpha-3 chain and its derived peptides GVR28, FFL25 and VTT30 is considerably enhanced with PLL as an intermediate layer as compared to the other parts of the collagen VI molecule.

FIG. 8: Quantitative evaluation of the keratinocyte adherence efficiency on titanium surfaces coated with different collagen VI peptides. Adherence of skin keratinocytes on the different titanium surfaces was assessed by scanning electron microscopy assays. The cell adherence efficiency with and without precoating with PLL was compared to the different collagen VI alpha-chains, and the bioactive collagen VI peptides GVR28, FYL25, FFL25, VTT30, and SFV33. Keratinocytes adherence mediated by the collagen VI alpha-3 chain and its derived peptides GVR28, FFL25 and VTT30 is considerably enhanced with PLL as an intermediate layer as an intermediate layer as compared to the other parts of the collagen VI molecule.

FIG. 9: Quantitative evaluation of the Gram-negative bacterial killing efficiency on collagen I scaffolds coated with different collagen VI peptides. Killing of Pseudomonas aeruginosa bacteria on collagen I scaffolds with 1 mm (thin biodegradable scaffold), or 2 mm (thick biodegradable scaffold) thickness, respectively, was assessed by scanning electron microscopy assays. The killing efficiency with and without precoating with PLL was compared to the different collagen VI alpha-chains, and the bioactive collagen VI peptides GVR28, FYL25, FFL25, VTT30, and SFV33. Killing by the collagen VI alpha-3 chain and its derived peptide SFV33 is considerably enhanced with PLL as an intermediate layer as compared to the other parts of the collagen VI molecule.

FIG. 10: Quantitative evaluation of the Gram-positive bacterial killing efficiency on collagen I scaffolds coated with different collagen VI peptides. Killing of Staphylococcus aureus bacteria on collagen I scaffolds with 1 mm (thin biodegradable scaffold), or 2 mm (thick biodegradable scaffold) thickness, respectively, was assessed by scanning electron microscopy assays. The killing efficiency with and without precoating with PLL was compared to the different collagen VI alpha-chains, and the bioactive collagen VI peptides GVR28, FYL25, FFL25, VTT30, and SFV33. Killing by the collagen VI alpha-3 chain and its derived peptide SFV33 is considerably enhanced with PLL as an intermediate layer as compared to the other parts of the collagen VI molecule.

FIG. 11: Quantitative evaluation of the fibroblast adherence efficiency on collagen I scaffolds coated with different collagen VI peptides. Adherence of skin fibroblasts on the different collagen I scaffolds was assessed by scanning electron microscopy assays. The cell adherence efficiency with and without precoating with PLL was compared to the different collagen VI alpha-chains, and the bioactive collagen VI peptides GVR28, FYL25, FFL25, VTT30, and SFV33. Fibroblast adherence mediated by the collagen VI alpha-3 chain and its derived peptides GVR28, FFL25 and VTT30 is considerably enhanced with PLL as an intermediate layer as compared to the other parts of the collagen VI molecule.

FIG. 12: Quantitative evaluation of the keratinocyte adherence efficiency on collagen I scaffolds surfaces coated with different collagen VI peptides. Adherence of skin keratinocytes on the different collagen I scaffolds was assessed by scanning electron microscopy assays. The cell adherence efficiency with and without precoating with PLL was compared to the different collagen VI alpha-chains, and the bioactive collagen VI peptides GVR28, FYL25, FFL25, VTT30, and SFV33. Keratinocytes adherence mediated by the collagen VI alpha-3 chain and its derived peptides GVR28, FFL25 and VTT30 is considerably enhanced with PLL as an intermediate as compared to the other parts of the collagen VI molecule.

### EXAMPLES

### Example 1

### Improved Binding of Collagen VI to a Solid Surface

Introduction

Biomaterials are placed internally to maintain or replace human body functions. They are constructed of various combinations of metal alloys, ceramics, polymers, or biopolymers due to their excellent mechanical properties, corrosion resistance, and biocompatibility. Wound matrices come in a variety of materials including natural polymers and synthetic polymers manufactured into various forms, such as foams, films, hydrocolloids, hydrogels, sponges, membranes, skin substitutes, electro spun micro- and nanofibers. Bioactive wound matrices deliver substances active in wound healing either by delivery of bioactive compounds or by being constructed from materials having endogenous activity. The healing success rate is highly determined by cellular and physiological processes taking place at the host-biomaterial interface during wound healing. Specifically, adverse host response processes often lead to chronic inflammation and encapsulation that preclude the performance of the biomaterial. Hence, it is important to design appropriate wound treatment strategies with the ability to work actively with wound properties such as tissues and cells to enhance healing. Also, patients often suffer from severe infections at the implant site, precluding the normal wound healing process. Usually, harmless bacteria like Staphylococci, Pseudomonas, or Streptococci can infiltrate the damaged tissue in the fresh wound. Here they may develop a high pathogenic potential and establish persistent infections, severely compromising biomaterial function. Therefore, new strategies including bioactive wound healing promoting biomaterial surfaces with antimicrobial effects can be beneficial for the patient. In particular, coating strategies, which allow loading a given biomaterial surface with a maximum quantity of bioactive biomolecules, are crucial for premium biomaterial function and biocompatibility.

To achieve this goal, the inventors investigated the effect of modifying titanium surfaces with Poly-L-Lysine (PLL) on the coating density of collagen VI and different peptides derived from its sequence. They were compared to a number of host defence molecules and peptides. Collagen VI and peptides thereof were immobilized to different collagen I-based biodegradable scaffold model discs on intermediate layers of poly-L-lysine (PLL) and exhibited an unexpected, particularly high coating efficiency, resembling native in vivo connective tissue structures.

In summary, these data show that all tested combinations of collagen VI/collagen VI peptide/PLL surface modifications exhibit particularly high coating efficiencies on the biomaterial surface. They may thus promote early and intermediate cellular events on the host-biomaterial interface at a particular high rate as compared to other biomolecules. Thus, collagen VI and its derived peptides, bound to an intermediate layer of PLL, may be considered biologically appropriate for premium biocompatibility and tissue integration of the bioactive host-biomaterial interface. In particular, they may protect the wound bed against local infections and the patient against systemic infections after biomaterial insertion and during the initial steps of wound closure and wound healing. This treatment strategy will be beneficial for the wound environment, with the potential to promote improved wound repair and reduce abnormal scar formation.

Therefore, in the present study, the inventors explored whether the use of PLL, together with native collagen VI-derived biomolecules, can promote superior coating density on the surface of different commercially available collagen scaffolds. Here, we describe for the first time that the use of polylysine such as PLL greatly enhances the surface coating of biological collagen scaffolds with native bioactive collagen VI molecules. This effect will result in a pronounced potential to provide a versatile, multifunctional, and appropriate extracellular environment, able to actively contrast the onset of infections and inflammation, while promoting tissue regeneration and scar remodeling, and consequently deliver the desired enhancement in biocompatibility.

Materials and Methods

Materials—MDS Collagen was obtained from MedSkin Solutions Dr. Suwelack A G (MDS). 1 mm thickness refers to thin, and 2 mm thickness refers to thick collagen scaffold, respectively. c-poly-L-lysine hydrobromide (PLL, 30 000-150 000 g/mol) was purchased from Sigma-Aldrich, St. Louis, USA. Collagen VI and its derived peptides were prepared as described in [18]. LL-37 was a kind gift of Dr. Artur Schmidtchen, University of Lund. NAT26, HKH20, GGL27 were kind gifts of Dr. Inga-Maria Frick, University of Lund. α defensins 3-6 and β defensins 1-4 were kind gifts of Dr. Arne Egesten, University of Lund. Proteins were radiolabeled with 131-iodine according to standard protocols prior to coating assays on collagen scaffold surfaces.

Biomaterial surface coating with PLL—collagen scaffold discs with a diameter of 5 mm and thickness of 2 mm (thick scaffold) and 1 mm (thin scaffold), respectively, were punched out from a larger sheet (approximately 10 cm×10 cm). Prior to collagen coating, 150 μl poly-L-lysine hydrobromide solution (0.2 mg/ml) were applied on the collagen scaffold discs by incubation at 60° C. for 2 h. Afterwards the discs were washed twice in distilled water to remove unbound PLL, air-dried and stored at room temperature.

Biomaterial surface coating with bioactive collagen VI molecules—collagen scaffold discs, after prior treatment with PLL, were put into 24-well cell culture plates (TPP, Trasadingen, Switzerland). They were coated by incubation with 150 μl bovine collagen VI microfibrils (concentration 15 μM), or with 150 μl collagen type VI peptides (concentration 3 μM), or with 150 ml LL-37, NAT26, HKH20, GGL27, α defensins 3-6 and β defensins 1-4 at 4° C. for 2 h, followed by rinsing with distilled water and air-drying. The coating efficiencies of the different biomolecules were assessed by determining the ratio between bound and free 131-iodine radioactivity associated with collagen scaffolds by determining radioactivity as cpm values in a γ-counter.

Results

PLL Mediates Superior Coating Efficiency of Collagen Type VI Microfibrils on Collagen Scaffolds.

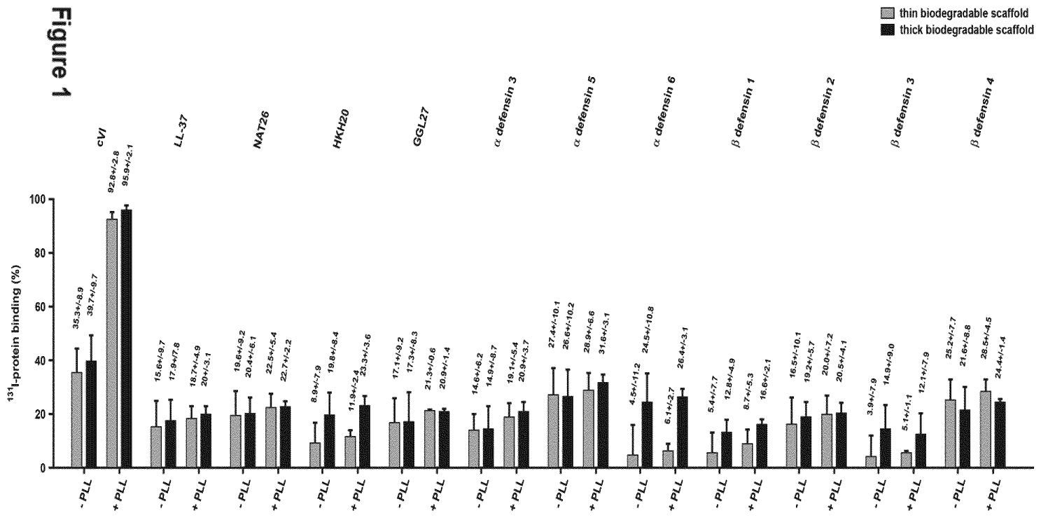

In order to assess possible effects of PLL on collagen type VI coating, collagen I scaffolds were pretreated with PLL. Coating with the cathelicidin peptide LL-37, and the host defence peptides and proteins NAT26, HKH20, GGL27, α defensins 3-6 and β defensins 1-4 served as controls. The results from 131-iodine assays show binding of the different proteins to collagen I scaffolds (FIG. 1). Pre-treatment of the collagen I scaffolds with PLL significantly enhanced the adherence of collagen VI microfibrils as compared to the controls (FIG. 1). Notably, the coating efficiency of collagen VI was considerably enhanced as compared to the previous gold standard human antimicrobial peptide LL-37.

The Collagen Type VI Binding Properties are Associated with the Alpha-3 Chain and the Bioactive Peptides Derived From it.

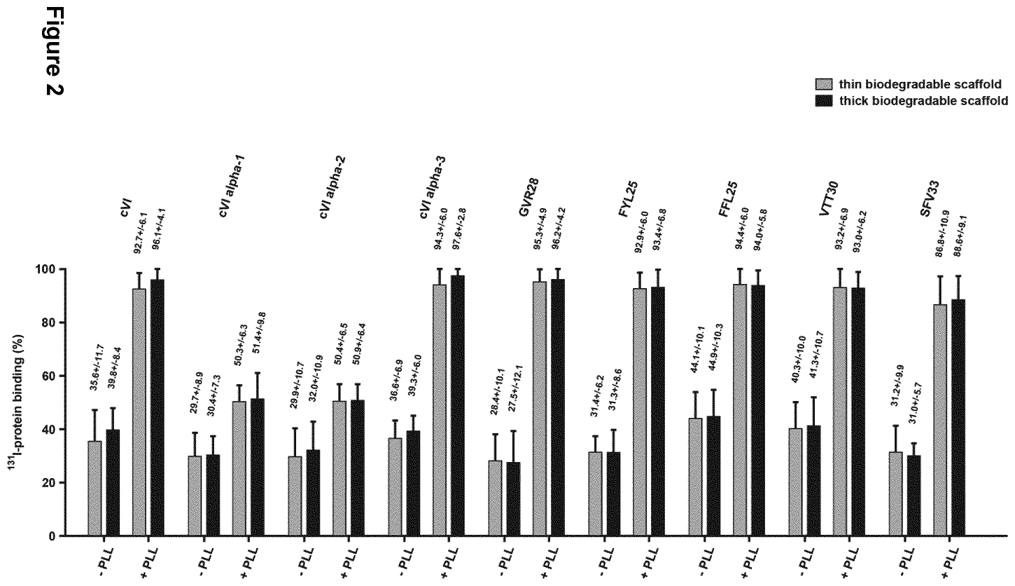

For a more detailed understanding of the underlying coating mechanism collagen I scaffolds were incubated with different collagen type VI-derived peptides in the presence or absence of PLL and assessed by 131-iodine radioactivity. FIG. 2 depicts the adherence of these molecules. In the presence of PLL superior coating of collagen I scaffolds with the collagen type VI alpha-3 chain, and the bioactive collagen VI peptides GVR28, FYL25, FFL25, VTT30, and SFV33 were observed.

Taken together, the data presented in FIGS. 1 and 2 show that collagen type VI and/or parts of the collagen VI alpha-3 chain strongly bind to collagen I scaffolds/biomaterials in the presence of PLL. This effect of PLL was considerably less pronounced for the other tested proteins. These effects are expected to result in superior wound healing properties of biomaterials coated with peptides derived from the collagen VI together with polylysine. This coating strategy will be beneficial for the wound environment, with the potential to promote improved wound repair and reduce abnormal scar formation.

### Example 2

### Improved Binding of Collagen Vi to a Titanium Surface

The inventors also investigated the effects of PLL on the binding of collagen VI directly to titanium surfaces.

Materials and methods

Materials—Titanium discs with a diameter and thickness of 5 mm and 0.25 mm, respectively, and with a maximum average roughness (Ra) of 0.8 μm, were punched out from a commercially available titanium foil (Alfa Aesar GmbH & Co. KG, Karlsruhe, Germany). ε-poly-L-lysine hydrobromide (PLL, 30,000-150,000 g/mol) was purchased from Sigma-Aldrich, St. Louis, USA. Collagen VI and its derived peptides were prepared as described in [18]. LL-37 was a kind gift of Dr. Artur Schmidtchen, University of Lund. NAT26, HKH20, GGL27 were kind gifts of Dr. Inga-Maria Frick, University of Lund. α defensins 3-6 and β defensins 1-4 were kind gifts of Dr. Arne Egesten, University of Lund. Proteins were radiolabeled with 131-iodine according to standard protocols prior to coating assays on collagen scaffold surfaces.

Biomaterial surface coating with PLL—Titanium discs were degreased with chloroform and 97% ethanol, followed by rinsing with distilled water, and air-drying at room temperature. Subsequently, prior to collagen coating, 150 μl poly-L-lysine hydrobromide (70,000-150,000 g/mol, Sigma-Aldrich, St. Louis, USA, 0.2 mg/ml) were applied on desired titanium discs by incubation at 60° C. for 2 hours. Afterwards, the titanium was washed twice in distilled water to remove unbound PLL, air-dried and stored at room temperature.

Biomaterial surface coating with bioactive collagen VI molecules—Titanium discs, after prior treatment with PLL, were put into 24-well cell culture plates (TPP, Trasadingen, Switzerland). They were coated by incubation with 150 μl collagen type VI peptides (concentration 3 μM), at 4° C. for 2 hours, followed by rinsing with distilled water and air-drying. The coating efficiencies of the different biomolecules were assessed by determining the ratio between bound and free 131-iodine radioactivity associated with titanium discs by determining radioactivity as cpm values in a γ-counter.

Results

PLL Mediates Superior Coating Efficiency of Collagen Type VI Microfibrils on Titanium Surfaces.

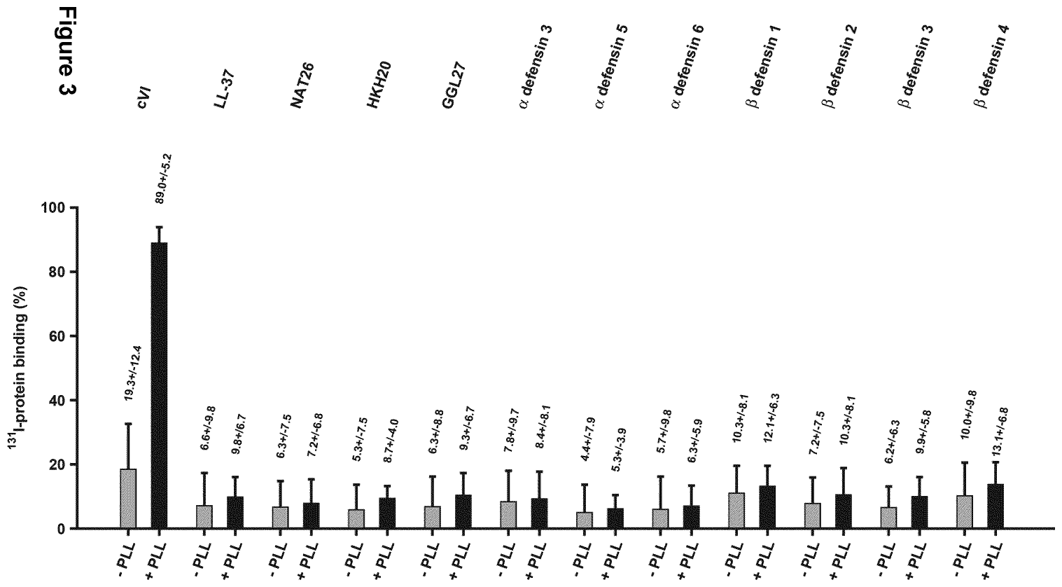

In order to assess possible effects of PLL on collagen type VI coating, titanium surfaces were pre-treated with PLL. Coating with the cathelicidin peptide LL-37, and the host defence peptides and proteins NAT26, HKH20, GGL27, α defensins 3-6 and β defensins 1-4 served as controls. The results from 131-iodine assays show binding of the different proteins to titanium surfaces (FIG. 3). Pre-treatment of the titanium surface with PLL significantly enhanced the adherence of collagen VI microfibrils as compared to the controls (FIG. 3). Notably, the coating efficiency of collagen VI was considerably enhanced as compared to the previous gold standard human antimicrobial peptide LL-37.

The Collagen Type VI Binding Properties are Associated with the Alpha-3 Chain and the Bioactive Peptides Derived from it.

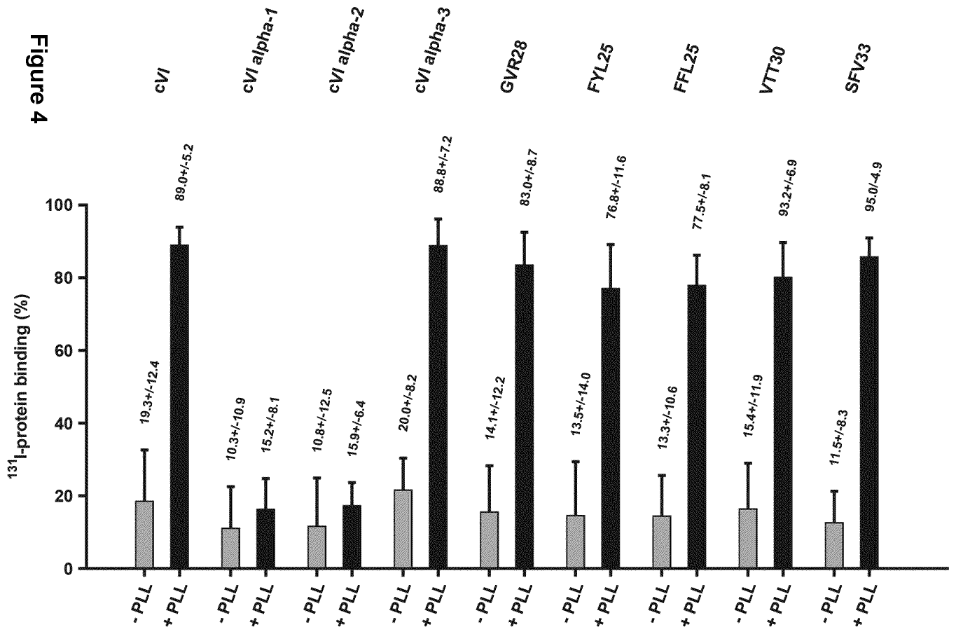

For a more detailed understanding of the underlying coating mechanism, titanium surfaces were also incubated with different collagen type VI-derived peptides in the presence or absence of PLL and assessed by 131-iodine radioactivity. FIG. 4 depicts the adherence of these molecules. In the presence of PLL superior coating of collagen I scaffolds with the collagen type VI alpha-3 chain, and the bioactive collagen VI peptides GVR28, FYL25, FFL25, VTT30, and SFV33 were observed.

Taken together, the data presented in FIGS. 3 and 4 show that collagen type VI and/or parts of the collagen VI alpha-3 chain also strongly bind to titanium surfaces in the presence of PLL. This effect of PLL was considerably less pronounced for the other tested proteins. These effects are expected to result in superior properties of titanium biomaterials coated with peptides derived from the collagen VI together with polylysine. This coating strategy will be beneficial for the use of titanium prosthesis, with the potential to promote improved wound repair and improved soft tissue integration of the prosthesis, as well as reduce infection risk due to accelerated wound closure at the host-titanium interface.

### Example 3

### Improved Bacterial Killing

Gram-negative and gram-positive bacterial killing efficiency of collagen I scaffolds and titanium surfaces coated with different collagen VI peptides with and without PLL was also investigated.

Collagen I scaffolds and titanium surfaces were coated with collagen VI microfibrils, each of collagen VI alpha 1-3 chains and the bioactive collagen VI peptides GVR28, FYL25, FFL25, VTT30, and SFV33 as described in Examples 1 and 2 above, respectively.

Killing efficiency of the gram-negative Pseudomonas aeruginosa bacteria and the gram-positive Staphylococcus aureus were assessed by scanning electron microscopy. For killing efficiency and scanning electron microscopy experiments, titanium scaffold discs with a size of 5 mm and a thickness of 0.25 mm, and collagen I scaffold discs with a size of 5 mm and a thickness of 1 mm (thin scaffold) or 2 mm (thick scaffold), respectively, were punched out and coated with PLL as described in Examples 1 and 2 above. They were subsequently incubated with 500 μL of bacterial suspension (5×10⁸ cfu/ml bacteria) and incubated for 2 hours at 37° C. in a humid atmosphere containing 5% CO₂.

The discs were then prepared for scanning electron microscopy. In short, samples were incubated overnight at 4° C. with fixation buffer (0.15M sodium cacodylate, pH 7.4, containing 2.5% v/v glutaraldehyde) and subsequently washed with cacodylate buffer (0.15M sodium cacodylate, pH 7.4), followed by a standard dehydration series with ethanol-water mixtures. Specimens were then dried in liquid CO₂, using ethanol as an intermediate solvent. Samples were mounted on aluminium discs and coated with 20 nm gold/palladium. Finally, samples were investigated with an XL 30 FEG scanning electron microscope and images were processed by AnalySIS ITEM software. Viable and non-viable bacteria were directly identified and quantified under in the scanning electron microscope due to their structural differences.

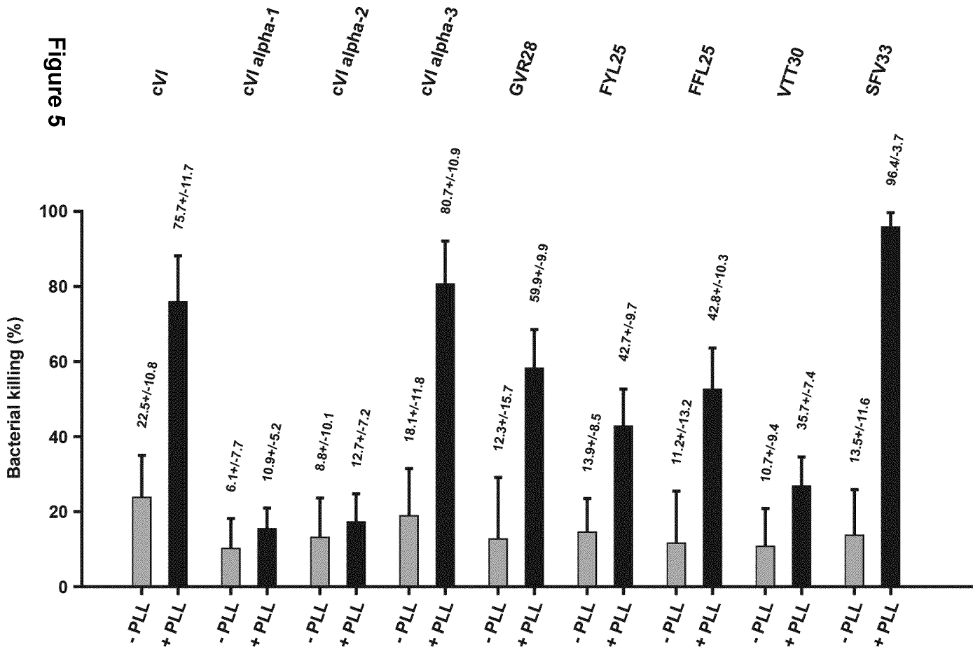

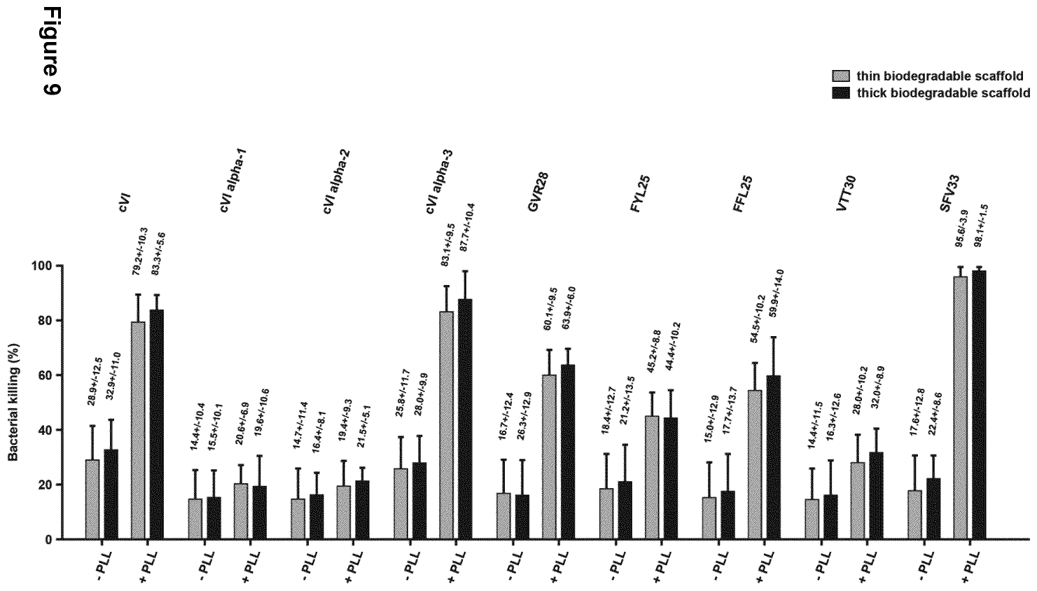

FIG. 5 (titanium surface) and FIG. 9 (collagen I scaffolds) demonstrate that killing of Pseudomonas aeruginosa is considerably enhanced by the collagen VI alpha-3 chain and its derived peptide SFV33 when PLL is used as an intermediate layer, compared to when PLL is not present, and compared to other parts of the collagen VI molecule.

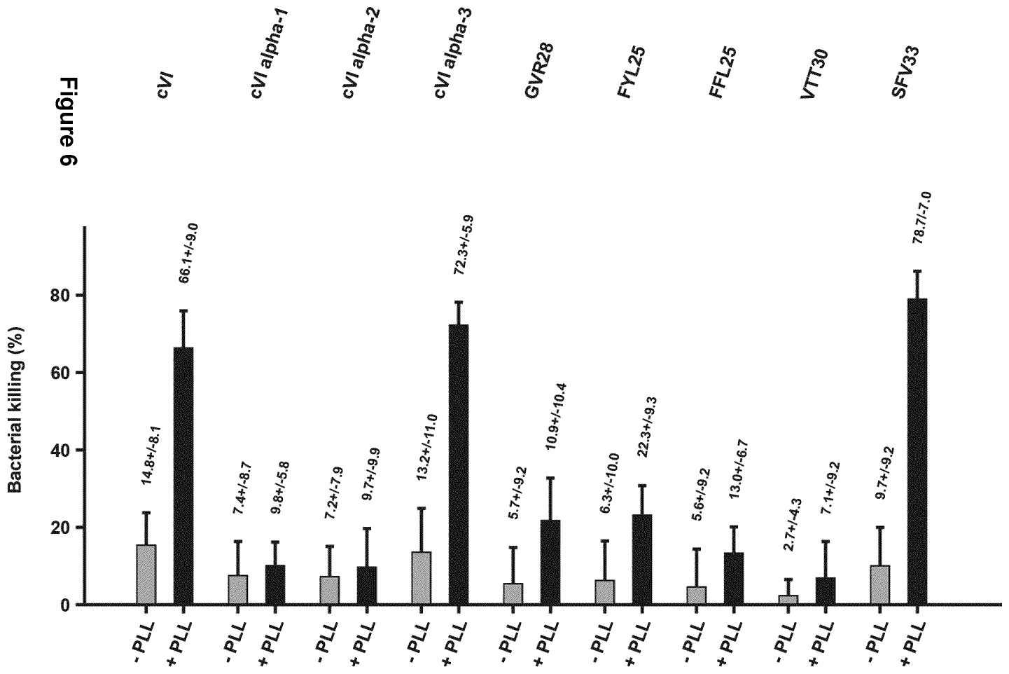

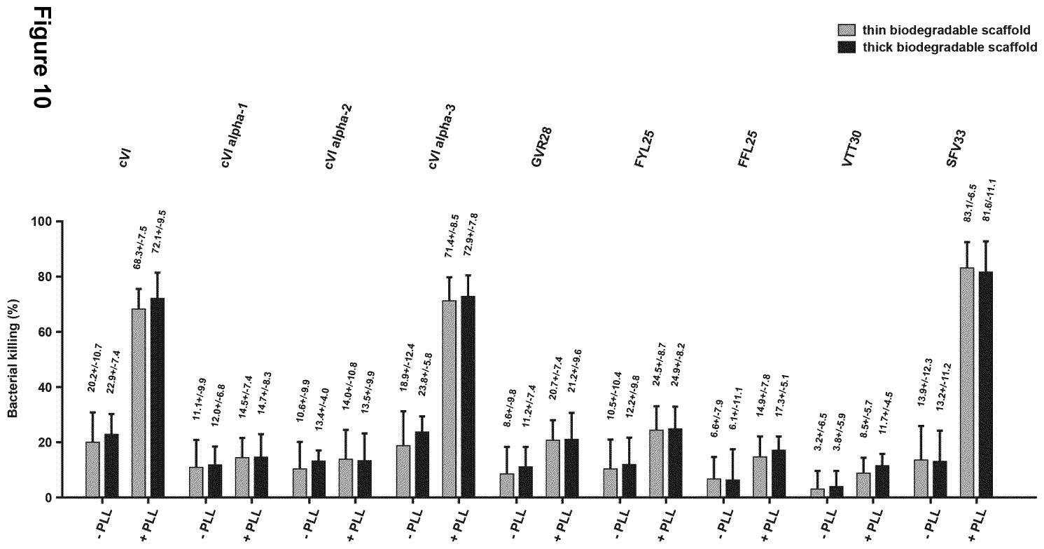

FIG. 6 (titanium surface) and FIG. 10 (collagen I scaffolds) demonstrate that killing of Staphylococcus aureus is considerably enhanced by the collagen VI alpha-3 chain and its derived peptide SFV33 when PLL is used as an intermediate layer, compared to when PLL is not present, and compared to other parts of the collagen VI molecule.

Taken together, these results demonstrate the ability of surfaces (both titanium surfaces and collagen I scaffolds) with enhanced collagen VI binding to achieve high levels of bacterial killing, and that this effect is applicable to both gram-positive and gram-negative bacteria. This confirms that the anti-microbial properties of collagen VI translate effectively to situations where collagen VI peptides are bound to surfaces utilising PLL.

### Example 4

### Improved Skin Cell Adherence During Wound Healing

Adherence of fibroblasts and keratinocytes to titanium surfaces coated with collagen VI peptides with and without polylysine was also investigated.

Collagen I scaffolds and titanium surfaces were coated with collagen VI microfibrils, each of collagen VI alpha 1-3 chains and the bioactive collagen VI peptides GVR28, FYL25, FFL25, VTT30, and SFV33 as described in Examples 1 and 2 above.

Adherence of fibroblasts or keratinocytes was assessed by scanning electron microscopy.

Cells and culture conditions—Keratinocytes (Human Epidermal Keratinocytes, adult (HEKa), C-005-5C, Gibco) and fibroblasts (Human Dermal Fibroblasts, adult (HDFa), C0135C, Gibco) were purchased from Thermo Fisher Scientific, Waltham, USA. Cryopreserved cells were thawed according to the manufacturer's protocol and seeded on three 75 mL tissue culture flasks, each containing 15 mL of keratinocyte basal medium (KBM; KBM Gold, Lonza Group AG, Basel, Switzerland). The medium was supplemented with transferrin, recombinant human epidermal growth factor (rhEGF), bovine pituitary extract (BPE), antibiotics (Gentamycin: GA-1000), insulin, epinephrine and hydrocortisone (PeproTech, New Jersey, USA). For fibroblasts, complete growth medium consisting of Dulbecco's modified eagle medium (D-MEM) with 100 mM sodium pyruvate (PAA Laboratories, Pasching, Austria), supplemented with 10% fetal bovine serum (FBS) (PAA Laboratories, Pasching, Austria) and 2 mM L-glutamine (Invitrogen, Carlsbad, USA), was used. Medium was changed every day and substituted with additional rhEGF at a final dilution of 1:1000. The cells were incubated for seven days until they reached confluency. The cells were harvested by trypsinization (TrypLE™ Select (1x), Life Technologies Corporation, Carlsbad, USA) and transferred into freezing medium, containing 1% BSA (Sigma Aldrich, St. Louis, USA) and 10% DMSO (Sigma-Aldrich, St. Louis, USA). 1 mL aliquots of the cell suspension were transferred into cryotubes. The cryotubes were transferred to the freezer at −80° C. for 24 hours and then finally stored in liquid nitrogen until further use.

Cell attachment—Cells were gently thawed and counted in a hemocytometer using trypan blue (Fisher Scientific, Waltham, USA) to estimate the number of live/dead cells. They were finally diluted in appropriate media to final cell concentrations of 16700 viable cells/mL. 300 μL cell solution were added on top of punched out titanium or collagen I scaffold samples, followed by incubation at 37° C. for 1 hour. Growth medium was removed and the cells were gently washed twice with PBS. The cells were then fixed by the addition of 4% formaldehyde in PBS, followed by incubation at 4° C. for 20 minutes. Finally, cells were rinsed three times in PBS and stored in 3 mL PBS until further evaluation by scanning electron microscopy.

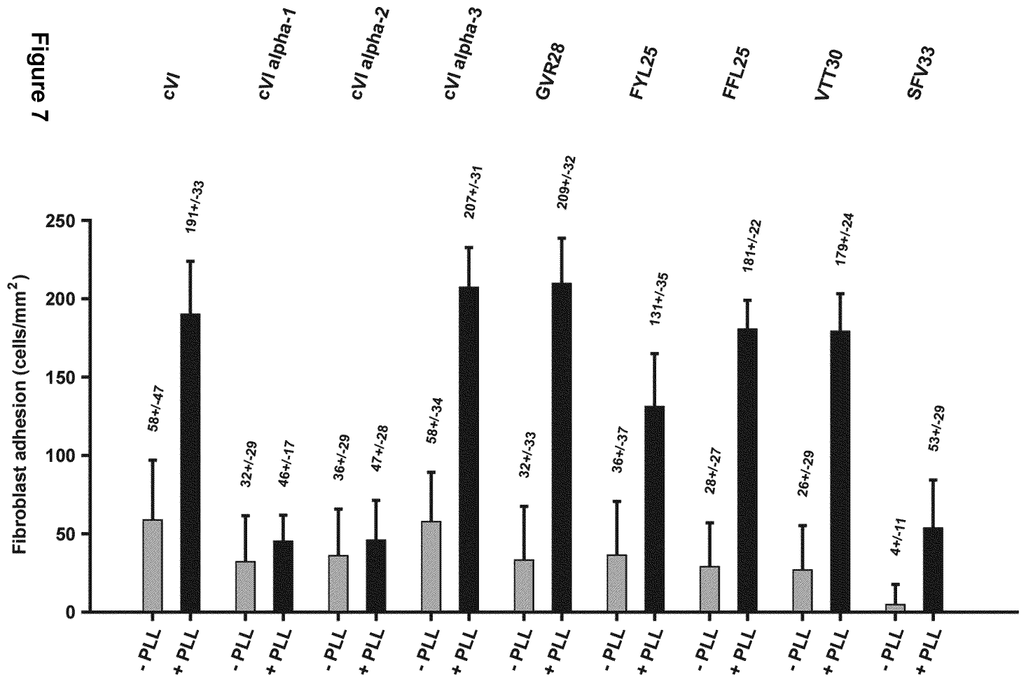

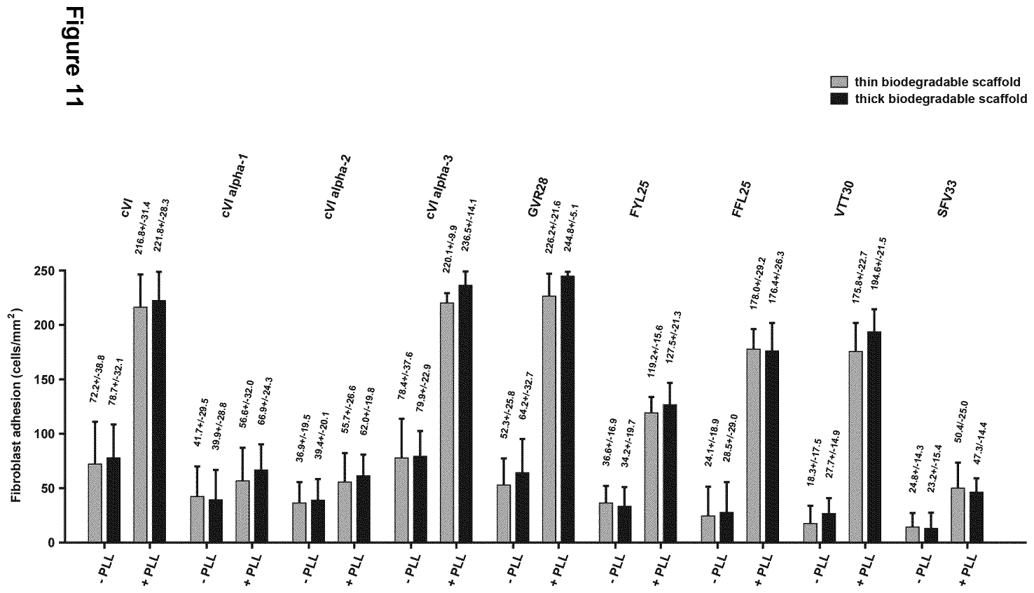

FIG. 7 (titanium surface) and FIG. 11 (collagen I scaffolds) demonstrate that fibroblast adherence (measured in cells/mm²) for the collagen VI alpha-3 chain and its derived peptides GVR28, FFL25 and VTT30 is considerably enhanced with PLL as an intermediate layer as compared to the other parts of the collagen VI molecule, and compared to when PLL is not used.

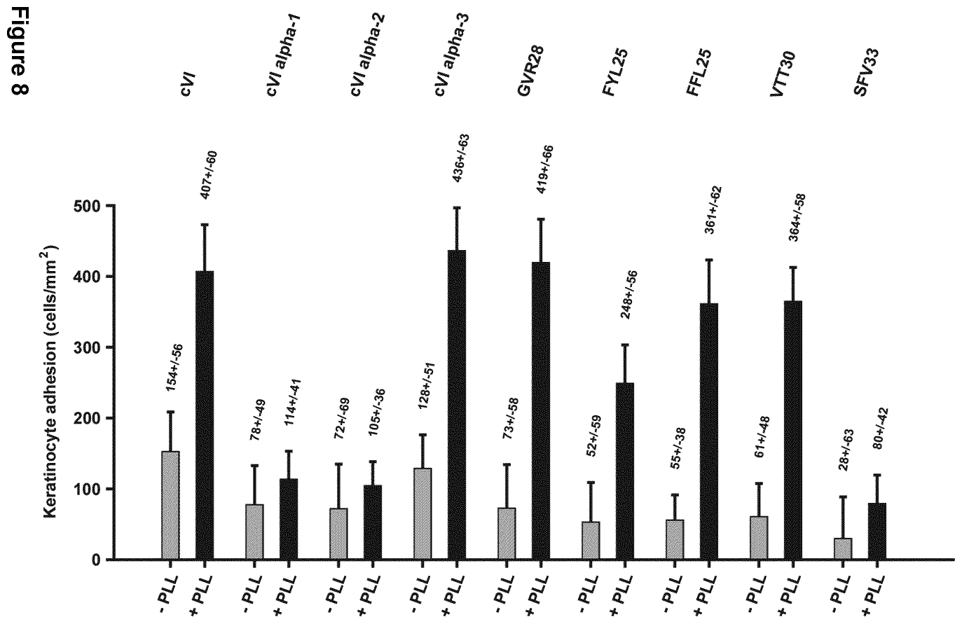

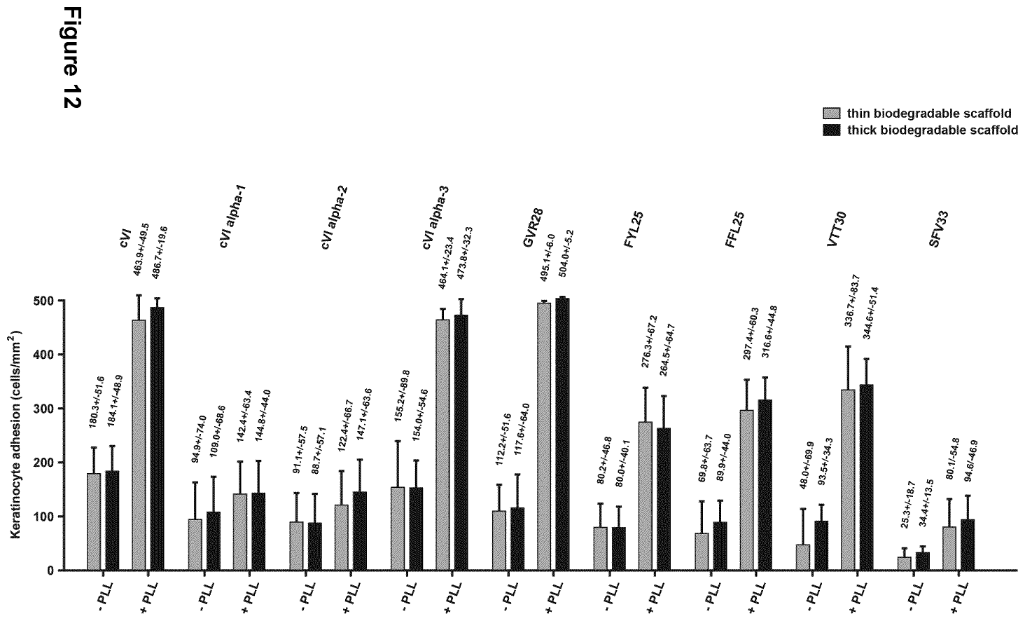

FIG. 8 (titanium surface) and FIG. 12 (collagen I scaffolds) demonstrate that keratinocyte adherence for collagen VI alpha-3 chain and its derived peptides GVR28, FFL25 and VTT30 is considerably enhanced with PLL as an intermediate layer as compared to the other parts of the collagen VI molecule, and compared to when PLL is not used.

Taken together, these results demonstrate the ability of surfaces (both titanium surfaces and collagen I scaffolds) coated with collagen VI alpha-3 chains and peptides derived from it to adhere to skin cells, for example keratinocytes and fibroblasts, with increased efficiency compared to surfaces bound to other parts of collagen VI, or surfaces bounds to collagen VI in the absence of PLL. This confirms the ability of collagen VI and its derived peptides to retain the ability to recruit skin cells when bound to surfaces in the presence of PLL, which will be beneficial in promoting the process of skin healing.

### REFERENCES

1. Gurtner G C, Werner S, Barrandon Y, Longaker M T. Wound repair and regeneration. Nature 2008; 453:314-21.
2. Sen C K, Gordillo G M, Roy S, Kirsner R, Lambert L, Hunt T K, Gottrup F, Gurtner G C, Longaker M T. Human skin wounds: a major and snowballing threat to public health and the economy. Wound Repair Regen 2009; 17:763-71.
3. Moura L I, Dias A M, Carvalho E, de Sousa H C. Recent advances on the development of wound dressings for diabetic foot ulcer treatment—-a review. Acta Biomater 2013; 9:7093-114.
4. Guo S, Dipietro L A. Factors affecting wound healing. J Dent Res 2010; 89:219-29.
5. Sen C K, Gordillo G M, Roy S, Kirsner R, Lambert L, Hunt T K, Gottrup F, Gurtner G C, Longaker M T. Human skin wounds: a major and snowballing threat to public health and the economy. Wound Repair Regen 2009; 17:763-71.
6. Shah J M Y, Omar E, Pai D R, Sood S. Cellular events and biomarkers of wound healing. Indian J Plast Surg 2012; 45:220-28.
7. Weller C, Sussman G. Wound dressings update. J Pharm Prac Res 2006; 36: 318-24.
8. Pereira R F, Barrias C C, Granja P L, Bartolo P J. Advanced biofabrication strategies for skin regeneration and repair. Nanomedicine (Lond) 2013; 8:603-21.
9. Martin, P. Wound healing—aiming for perfect skin regeneration. Science 1997; 276:75-81.
10. Kirby, G. T., Mills, S. J., Cowin, A. J., Smith, L. E. Stem cells for cutaneous wound healing. Biomed Res Int 2015; 2015:285869.
11. Isakson M, de Blacam C, Whelan D, McArdle A, Clover A J. Mesenchymal stem cells and cutaneous wound healing: current evidence and future potential. Stem Cells Int 2015; 2015:831095.
12. Sinno H, Prakash S. Complements and the wound healing cascade: an updated review. Plast Surg Int 2013; 2013:146764.
13. Wound healing: an overview. Broughton G 2nd, Janis J E, Attinger C E. Plast Reconstr Surg 2006; 117(7 Suppl): 1e-S-32e-S.
14. Das S, Baker A B. Biomaterials and Nanotherapeutics for Enhancing Skin Wound Healing. Front Bioeng Biotechno 2016; 4:82. eCollection 2016.
15. Hunt T K. The physiology of wound healing. Ann Emerg Med 1988; 17:1265-73
16. Abou Neel E A, Bozec L, Knowles J C, Syed O, Mudera V, Day R, Hyun J K. Collagen—emerging collagen based therapies hit the patient. Adv Drug Deliv Rev 2013; 65:429-56.
17. An B, Lin Y S, Brodsky B. Collagen interactions: Drug design and delivery. Adv Drug Deliv Rev 2016; 97:69-84.
18. Abdillahi S M, Maaß T, Kasetty G, Strömstedt A A, Baumgarten M, Tati R, Nordin S L, Walse B, Wagener R, Schmidtchen A, Mörgelin M. Collagen VI Contains Multiple Host Defense Peptides with Potent In Vivo Activity. J Immunol 2018; 201:1007-1020.
19. Specks, U., Mayer, U., Nischt, R., Spissinger, T., Mann, K., Timpl, R., Engel, J., and Chu, M. L. (1992) Structure of recombinant N-terminal globule of type VI collagen alpha 3 chain and its binding to heparin and hyaluronan. The EMBO journal 11, 4281-4290
20. Chu, M. L., Pan, T. C., Conway, D., Kuo, H. J., Glanville, R. W., Timpl, R., Mann, K., and Deutzmann, R. (1989) Sequence analysis of alpha 1(VI) and alpha 2(VI) chains of human type VI collagen reveals internal triplication of globular domains similar to the A domains of von Willebrand factor and two alpha 2(VI) chain variants that differ in the carboxy terminus. The EMBO journal 8, 1939-1946
21. Chu, M. L., Zhang, R. Z., Pan, T. C., Stokes, D., Conway, D., Kuo, H. J., Glanville, R., Mayer, U., Mann, K., Deutzmann, R., and et al. (1990) Mosaic structure of globular domains in the human type VI collagen alpha 3 chain: similarity to von Willebrand factor, fibronectin, actin, salivary proteins and aprotinin type protease inhibitors. The EMBO journal 9, 385-393
22. Chu, M. L., Pan, T. C., Conway, D., Saitta, B., Stokes, D., Kuo, H. J., Glanville, R. W., Timpl, R., Mann, K., and Deutzmann, R. (1990) The structure of type VI collagen. Annals of the New York Academy of Sciences 580, 55-63
23. Lamande, S. R., Mörgelin, M., Adams, N. E., Selan, C., and Allen, J. M. (2006) The C5 domain of the collagen type VI alpha3(VI) chain is critical for extracellular microfibril formation and is present in the extracellular matrix of cultured cells. The Journal of Biological Chemistry 281, 16607-16614
24. Cescon, M., Gattazzo, F., Chen, P., and Bonaldo, P. (2015) Collagen VI at a glance. Journal of Cell Science 128, 3525-3531
25. Collagen-based biomaterials for tissue engineering applications. Parenteau-Bareil R., Gauvin R., Berthod F. Materials 2010; 3:1863-87.
26. Chattopadhyay S, Raines R T. Review collagen-based biomaterials for wound healing. Biopolymers 2014; 101:821-33.
27. Baum C L, Arpey C J. Normal cutaneous wound healing: clinical correlation with cellular and molecular events. Dermatol Surg 2005; 31:674-86.
28. Ghica M V, Albu Kaya M G, Dinu-Pirvu C E, Lupuleasa D, Udeanu D I. Development, Optimization and In Vitro/In Vivo Characterization of Collagen-Dextran Spongious Wound Dressings Loaded with Flufenamic Acid. Molecules 2017; 22:1552.
29. Guo R, Lan Y, Xue W, Cheng B, Zhang Y, Wang C, Ramakrishna S. Collagen-cellulose nanocrystal scaffolds containing curcumin-loaded microspheres on infected full-thickness burns repair. J Tissue Eng Regen Med 2017; 11:3544-55.
30. Bhowmick S., Thanusha A. V., Kumar A., Scharnweber D., Rother S., Koul V. (2018). Nanofibrous artificial skin substitute composed of mPEG—PCL grafted gelatin/hyaluronan/chondroitin sulfate/sericin for 2nd degree burn care: in vitro and in vivo study. RSC Adv 2018; 8:16420-32.
31. Yoon D, Yoon D, Cha H J, Lee J S, Chun W. Enhancement of wound healing efficiency mediated by artificial dermis functionalized with EGF or NRG1. Biomed Mater 2018; 13:045007.
32. Powell H M, Supp D M, Boyce S T. Influence of electrospun collagen on wound contraction of engineered skin substitutes. Biomaterials 2008; 29:834-43.
33. Hall Barrientos I J, Paladino E, Szabó P, Brozio S, Hall P J, Oseghale C I, Passarelli M K, Moug S J, Black R A, Wilson C G, Zelkó R, Lamprou D A. Electrospun collagen-based nanofibres: A sustainable material for improved antibiotic utilisation in tissue engineering applications. Int J Pharm 2017; 531:67-79.
34. Golser A. V., Ro M., Bo H. G., Scheibel T. Engineered collagen: a redox switchable framework for tunable assembly and fabrication of biocompatible surfaces. ACS Biomater Sci Eng 2018; 4:2106-14.
35. Velnar T, Bailey T, Smrkolj V. The wound healing process: an overview of the cellular and molecular mechanisms. J Int Med Res 2009; 37:1528-42.
36. Davis J R (Ed.) Handbook of Materials for Medical Devices, 4th ed.; ASM International: Novelty, OH, USA, 2006
37. Lewallen E A, Riester S M, Bonin C A, Kremers H M, Dudakovic A, Kakar S, Cohen R C, Westendorf J J, Lewallen D G, van Wijnen A J. Biological strategies for improved osseointegration and osteoinduction of porous metal orthopedic implants. Tissue Eng. Part B Rev. 2015:21; 218-230
38. Geeth, M, Singh A K, Asokamani R, Gogia A K. Ti Based Biomaterials, the Ultimate Choice for Orthopaedic Implants—A Review. Prog Mater Sci. 2009; 54:397-425
39. Welsink C L, Lambers K T A, van Deurzen D F P, Eygendaal D, van den Bekerom M P J. Total Elbow Arthroplasty: A Systematic Review. JBJS Rev. 2017; 5:e4
40. Lou M, Li N, Xia L. Pedicle Screw Versus Hybrid Instrumentation in Adolescent Idiopathic Scoliosis: A Systematic Review and Meta-analysis with Emphasis on Complications and Reoperations. Medicine (Baltimore). 2017; 96:e7337
41. Bahraminasab M, Farahmand F. State of the Art Review on Design and Manufacture of Hybrid Biomedical Materials: Hip and Knee Prostheses.Proc Inst Mech Eng H. 2017; 231:785-813
42. Petrillo S, Longo U G, Papalia R, Denaro V. Reverse Shoulder Arthroplasty for Massive Irreparable Rotator Cuff Tears and Cuff Tear Arthropathy: A Systematic Review. Musculoskelet Surg. 2017; 101:105-112
43. Ige O O, Umoru L E, Aribo S. Natural Products: A Minefield of Biomaterials. ISRN Mater Sci 2012; 2012:983062:1-983062:20
44. Lu J Z, Wu L J, Sun G F, Luo K Y, Zhang Y K, Cai J, Cui C Y, Luo X M. Microstructural Response and Grain Refinement Mechanism of Commercially Pure Titanium Subjected to Multiple Laser Shock Peening Impacts. Acta Mater. 2017; 127:252-266
45. Hong D, Chou D-T, Velikokhatnyi O I, Roy A, Lee B, Swink I, lssaev I, Kuhn H A, Kumta P N. Binder-jetting 3D Printing and Alloy Development of New Biodegradable Fe—Mn−Ca/Mg Alloys. Acta Biomater. 2016; 45:375-386
46. Trivedi P, Nune K C, Misra R D K, Goel S, Jayganthan R, Srinivasan A. Grain Refinement to Submicron Regime in Multiaxial Forged Mg-2Zn-2Gd Alloy and Relationship to Mechanical Properties. Mater Sci Eng A. 2016; 668:59-65
47. Niinomi M, Narushima T, Nakai M. Advances in Metallic Biomaterials; Springer: Berlin/Heidelberg, Germany, 2015
48. Niinomi M. Mechanical Biocompatibilities of Titanium Alloys for Biomedical Applications. J Mech Behav Biomed Mater. 2008; 1:30-42
49. Sul Y T, Johansson C B, Petronis S, Krozer A, Jeong Y, Wennerberg A, Albrektsson T. Characteristics of the Surface Oxides on Turned and Electrochemically Oxidized pure Titanium Implants up to Dielectric Breakdown: the Oxide Thickness, Micropore Configurations, Surface Roughness, Crystal Structure and Chemical Composition. Biomaterials. 2002; 23:491-501
50. De Vasconcellos L M R, Rodarte Y, do Prado R F, de Vasconcellos L G O, Alencastro Graa M L D, Alves Cairo C A. Porous Titanium by Towder Metallurgy for Biomedical Application: Characterization, Cell Cytotoxity and in vivo Tests of Osseointegration. In Biomedical Engineering—Technical Applications in Medicine; Hudak, R., Ed.; InTech: Rijeka, Croatia, 2012
51. Niinomi, M.; Nakai, M.; Hieda, J. Development of new Metallic Alloys for Biomedical Applications. Acta Biomater. 2012; 8:3888-3903
52. Cremasco A, Messias A D, Esposito A R, Eliana Aparecida de Rezende D, Caram R. Effects of Alloying Elements on the Cytotoxic Response of Titanium Alloys. Mater Sci Eng C. 2011; 31:833-839
53. Goyenvalle E, Aguado E, Cognet R, Bourges X, Daculsi G. Calcium Phosphate Ceramic Blasting on Titanium Surface Improve Bone Ingrowth. KEM. 2008; 361:1351-1354
54. Chan C W, Hussain I, Waugh DG, Lawrence J, Man H C. Effect of Laser Treatment on the Attachment and Viability of Mesenchymal Stem Cell Responses on Shape Memory NiTi Alloy. Mater Sci Eng C. 2014; 42:254-263
55. Fage S W, Muris J, Jakobsen S S, Thyssen J P. Titanium: A Review on Exposure, Release, Penetration, Allergy, Epidemiology, and Clinical Reactivity. Contact Dermat. 2016; 74:323-345
56. Tabish T A, Butt M T, Ali M. Biocompatibility Behavior and Biomedical Applications of Ti—Ni Based Shape Memory Alloys: A Brief Review. J Fac Eng Technol. 2006; 19:135-140
57. Ratner B D, Hoffman A S, Schoen F J, Lemons J E. Biomaterials Science: An Introduction to Materials in Medicine; Elsevier Academic Press: New York, N.Y., USA, 2004
58. Mahtabi M J, Shamsaei N, Mitchell M R. Fatigue of Nitinol: The State-of-the-art and Ongoing Challenges. J Mech Behav Biomed Mater. 2015; 50:228-254
59. Meredith N. Assessment of Implant Stability as a Prognostic Determinant. Int J Prosthodont. 1998; 11:491-501

1. A composition comprising:
(a) collagen type VI, or a polypeptide comprising or consisting of an amino acid sequence derived from collagen type VI, or a fragment, variant, fusion or derivative thereof, or a fusion of said fragment, variant of derivative thereof; and
(b) polylysine.

2. A composition according to claim 1, wherein the polylysine is poly-L-lysine.

3. A composition according to any one of claim 1 or 2, wherein the collagen type VI or polypeptide, fragment, variant, fusion or derivative is capable of killing or attenuating the growth of microorganisms.

4. A composition according to claim 3 wherein the microorganisms are selected from the group consisting of bacteria, mycoplasmas, yeasts, fungi and viruses.

5. A composition according to any one of the preceding claims wherein the collagen type VI or polypeptide is capable of binding to the membrane of the microorganism.

6. A composition according to any one of the preceding claims wherein the collagen type VI or polypeptide is capable of causing membrane disruption of the microorganisms.

7. A composition according to any one of the preceding claims which is capable of promoting wound closure.

8. A composition according to any one of the preceding claims, wherein the collagen type VI or polypeptide is capable of exhibiting an antimicrobial effect greater than or equal to that of LL-37.

9. A composition according to any one of the preceding claims wherein the microorganisms are Gram-positive or Gram-negative bacteria.

10. A composition according to claim 9, wherein the microorganisms are selected from the group consisting of: Pseudomonas aeruginosa, Staphylococcus aureus, Escherichia coli, group A Streptococcus (e.g. Streptococcus pyogenes), group B Streptococcus (e.g. Streptococcus agalactiae), group C Streptococcus (e.g. Streptococcus dysgalactiae), group D Streptococcus (e.g. Enterococcus faecalis), group F Streptococcus (e.g. Streptococcus anginosus), group G Streptococcus (e.g. Streptococcus dysgalactiae equisimilis), alpha-hemolytic Streptococcus (e.g. Streptococcus viridans, Streptococcus pneumoniae), Streptococcus bovis, Streptococcus mitis, Streptococcus anginosus, Streptococcus sanguinis, Streptococcus suis, Streptococcus mutans, Moraxella catarrhalis, Non-typeable Haemophilus influenzae (NTHi), Haemophilus influenzae b (Hib), Actinomyces naeslundii, Fusobacterium nucleatum, Prevotella intermedia, Klebsiella pneumoniae, Enterococcus cloacae, Enterococcus faecalis, Staphylococcus epidermidis, multi-resistant Pseudomonas aeruginosa (MRPA), multi-resistant Staphylococcus aureus (MRSA), multi-resistant Escherichia coli (MREC), multi-resistant Staphylococcus epidermidis (MRSE) and multi-resistant Klebsiella pneumoniae (MRKP).

11. A composition according to any one of the preceding claims wherein the microorganisms are bacteria which are resistant to one or more conventional antibiotic agents.

12. A composition according to claim 11 wherein the microorganism is selected from the group consisting of: multidrug-resistant Staphylococcus aureus (MRSA), multidrug-resistant Pseudomonas aeruginosa (MRPA), multi-resistant Escherichia coli (MREC), multi-resistant Staphylococcus epidermidis (MRSE) and multi-resistant Klebsiella pneumoniae (MRKP).

13. A composition according to any one of the preceding claims wherein the collagen type VI or polypeptide is substantially non-toxic to mammalian cells.

14. A composition according to any one of the preceding claims wherein the collagen type VI or polypeptide is capable of exerting an anti-endotoxic effect.

15. A composition according to any one of the preceding claims wherein the polypeptide is derived from a von Willebrand Factor type A domain.

16. A composition according to any one of the preceding claims wherein the polypeptide is or is derived from the α1, α2 and/or α3 chain of collagen type VI.

17. A composition according to claim 16 wherein the polypeptide is or is derived from the α3 chain of collagen type VI.

18. A composition according to any one of the preceding claims wherein the polypeptide is or is derived from the N2, N3 or C1 domain of the α3 chain of collagen type VI.

19. A composition according to any one of the preceding claims wherein the collagen type VI or polypeptide has a net positive charge.

20. A composition according to claim 19 wherein the charge on the collagen type VI or polypeptide ranges from between +2 to +9.

21. A composition according to any one of the preceding claims wherein the collagen type VI or polypeptide has at least 30% hydrophobic residues.

22. A composition according to any one of the preceding claims comprising or consisting of the amino acid sequence selected from the group consisting of SEQ ID NOs: 1 to 23, and fragments, variants, fusions or derivatives thereof, and fusions of said fragments, variants and derivatives thereof, which retain an antimicrobial activity of any one of SEQ ID NOs:1 to 23.

23. A composition according to claim 22 comprising or consisting of the amino acid sequence selected from the group consisting of SEQ ID NOs: 1 to 5:
[SEQ ID NO: 1]
“GVR28”: GVRPDGFAHIRDFVSRIVRRLNIGPSKV
[SEQ ID NO: 2]
“FYL25”: FYLKTYRSQAPVLDAIRRLRLRGGS
[SEQ ID NO: 3]
“FFL25”: FFLKDFSTKRQIIDAINKVVYKGGR
[SEQ ID NO: 4]
“VTT30”: VTTEIRFADSKRKSVLLDKIKNLQVALTSK
[SEQ ID NO: 5]
“SFV33”: SFVARNTFKRVRNGFLMRKVAVFFSNTPTRASP
and fragments, variants, fusions or derivatives thereof, and fusions of said fragments, variants and derivatives thereof, which retain an antimicrobial activity of any one of SEQ ID NOs:1 to 5.

24. A composition according to claim 23 comprising or consisting of the amino acid sequence selected from the group consisting of SEQ ID NOs: 1 to 5.

25. A composition according to any one of the preceding claims wherein the polypeptide comprises or consists of a variant of the amino acid sequence selected from the group consisting of SEQ ID NOs: 1 to 23.

26. A composition according to claim 25 wherein the variant has at least 50% identity with the amino acid sequence amino acid sequence of any one of SEQ ID NOs: 1 to 23, for example at least 55%, 60%, 65%, 70%, 75%, 80%, 85%, 90%, 95%, 96%, 97%, 98% or at least 99% identity.

27. A composition according to any one of the preceding claims wherein the polypeptide is between 10 and 200 amino acids in length, for example between 10 and 150, 15 and 100, 15 and 50, 20 and 40 or 25 and 35 amino acids in length.

28. A composition according to claim 27 wherein the polypeptide is at least 20 amino acids in length.

29. A composition according to any one of the preceding claims wherein the collagen type VI or polypeptide, or fragment, variant, fusion or derivative thereof, comprises one or more amino acids that are modified or derivatised.

30. A composition according to claim 29 wherein the one or more amino acids are modified or derivatised by PEGylation, amidation, esterification, acylation, acetylation and/or alkylation.

31. A composition according to any one of the preceding claims wherein the collagen type VI or polypeptide is a recombinant polypeptide.

32. A composition according to any one of claims 1 to 31, wherein the polylysine is polymerized at the ε carbon position of the lysine monomer units.

33. A composition according to any one of claims 1 to 32, wherein the polylysine has a molecular weight in the range 30,000 Da to 300,000 Da, preferably wherein the polylysine has a molecular weight of 30,000 Da to 70,000 Da.

34. A composition according to any one of claims 1 to 33 wherein the polylysine is made up of between 200 and 2054 monomer units of lysine polymerized together, preferably wherein the polylysine is made up of between 200 and 480 monomer units of lysine polymerized together.

35. A pharmaceutical composition comprising a composition according to any one of claims 1 to 34 together with a pharmaceutically acceptable excipient, diluent, carrier, buffer or adjuvant.

36. A medical device, implant, wound care product, or material for use in the same, which is coated, impregnated, admixed or otherwise associated with a composition or pharmaceutical composition according to any one of the preceding claims.

37. A medical device, implant, wound care product, or material for use in the same according to claim 36, which is coated with a composition according to any one of claims 1 to 34 or pharmaceutical composition according to claim 35.

38. A medical device, implant, wound care product, or material for use in the same, according to any one of claim 36 or 37 wherein the device, implant, wound care product, or material is for use in by-pass surgery, extracorporeal circulation, wound care and/or dialysis.

39. A medical device, implant, wound care product, or material for use in the same, according to any one of claims 36 to 38 wherein the composition is coated, painted, sprayed or otherwise applied to a suture, prosthesis, implant, wound dressing, catheter, lens, skin graft, skin substitute, fibrin glue or bandage.

40. A medical device, implant, wound care product, or material for use in the same, according to any one of claims 36 to 39 comprising or consisting of a polymer, metal, metal oxide and/or ceramic.

41. A medical device, implant, wound care product, or material for use in the same according to claim 40, comprising or consisting of a metal, wherein the metal is titanium or a titanium alloy.

42. A medical device, implant, wound care product, or material for use in the same according to any one of claims 36 to 41, wherein the device, implant, wound care product, or material is for use in the same is selected from the group consisting of: a joint prosthesis, an orthopaedic device, a bone replacement device, a bone fixation device, a bone plate, a stem of an artificial hip joint, an artificial organ, a spinal rod, a maxillofacial plate, a stent graft, a percutaneous device, and a pacemaker.

43. A medical device, implant, wound care product, or material for use in the same according to any one of claims 36 to 42 wherein the percentage binding of collagen type VI or the polypeptide of the composition to the medical device, implant, wound care product, or material for use in the same is at least 50%, at least 60%, at least 70%, at least 80%, at least 85%, or at least 86.8%.

44. A medical device, implant, wound care product, or material for use in the same, according to any one of claims 36 to 43 wherein the percentage binding of collagen type VI or the polypeptide of the composition to the medical device, implant, wound care product, or material for use in the same is greater than the percentage binding achieved by a composition that does not contain polylysine, for example; at least 10%, 20%, 30%, 40%, 50%, 60%, 70%, 80%, 90%, 100%, or at least 109% greater.

45. A method of preparing a medical device, implant, wound care product or material for use in the same, comprising the step of coating, impregnating, admixing or otherwise associating the medical device, implant, wound care product, or material for use in the same with the composition or pharmaceutical composition according to any one of claims 1 to 35.

46. A method of preparing a medical device, implant, wound care product or material for use in the same comprising the following steps:
(i) preparing a composition comprising collagen type VI or a polypeptide as defined by any one of claims 1 and 3 to 31 and polylysine as defined by any one of claims 1 to 2 and 32 to 34; and
(ii) coating, impregnating, admixing or otherwise associating the medical device, implant, wound care product, or material for use in the same with the composition prepared in step (i).

47. A method of preparing a medical device, implant, wound care product or material for use in the same comprising the following steps:
(i) coating, impregnating, admixing or otherwise associating the medical device, implant, wound care product, or material for use in the same with polylysine as defined in any one of claims 1 to 2 and 32 to 34; and
(ii) coating, impregnating, admixing or otherwise associating the medical device, implant, wound care product, or material for use in the same produced in step (i) with collagen type VI or a polypeptide as defined in any one of claims 1 and 3 to 32.

48. A kit comprising:
(i) a composition according to any one of claims 1 to 34 or a pharmaceutical composition according to claim 35 or a medical device, implant, wound care product, or material for use in the same according to any one of claims 36 to 44, and
(ii) instructions for use.

49. A kit comprising:
(i) a collagen type VI or polypeptide as defined in any one of claims 1 and 3 to 31;
(ii) polylysine as defined in any one of claims 1 to 2 and 32 to 34; and
(iii) instructions for use.

50. A composition according to any one of claims 1 to 34 or a pharmaceutical composition as defined in claim 35 for use in medicine.

51. A composition according to any one of claims 1 to 34 or a pharmaceutical composition as defined in claim 35 for use in the curative and/or prophylactic treatment of microbial infections.

52. A composition or pharmaceutical composition for use according to claim 51 wherein the microbial infection is a systemic infection.

53. A composition or pharmaceutical composition for use according to claim 51 or 52 wherein the microbial infection is resistant to one or more conventional antibiotic agents.

54. A composition or pharmaceutical composition for use according to any one of claims 51 to 53 wherein the microbial infection is caused by a microorganism selected from the group consisting of: Pseudomonas aeruginosa, Staphylococcus aureus, Escherichia coli, group A Streptococcus (e.g. Streptococcus pyogenes), group B Streptococcus (e.g. Streptococcus agalactiae), group C Streptococcus (e.g. Streptococcus dysgalactiae), group D Streptococcus (e.g. Entero-coccus faecalis), group F Streptococcus (e.g. Streptococcus anginosus), group G Streptococcus (e.g. Streptococcus dysgalactiae equisimilis), alpha-hemolytic Streptococcus (e.g. Streptococcus viridans, Streptococcus pneumoniae), Streptococcus bovis, Streptococcus mitis, Streptococcus anginosus, Streptococcus sanguinis, Streptococcus suis, Streptococcus mutans, Moraxella catarrhalis, Non-typeable Haemophilus influenzae (NTHi), Haemophilus influenzae b (Hib), Actinomyces naeslundii, Fusobacterium nucleatum, Prevotella intermedia, Klebsiella pneumoniae, Enterococcus cloacae, Enterococcus faecalis, Staphylococcus epidermidis, multi-resistant Pseudomonas aeruginosa (MRPA), and multi-resistant Staphylococcus aureus (MRSA), multi-resistant Escherichia coli (MREC), multi-resistant Staphylococcus epidermidis (MRSE) and multi-resistant Klebsiella pneumoniae (MRKP).

55. A composition or pharmaceutical composition for use according to any one of claims 51 to 54 wherein the microbial infection is caused by a microorganism selected from the group consisting of: multidrug-resistant Staphylococcus aureus (MRSA) and multidrug-resistant Pseudomonas aeruginosa (MRPA).

56. A composition or pharmaceutical composition for use according to any one of claims 50 to 55 in combination with one or more additional antimicrobial agents.

57. A composition or pharmaceutical composition for use according to claim 56 wherein the one or more additional antimicrobial agent is selected from the group consisting of: antimicrobial polypeptides and antibiotics.

58. A composition as defined in any one of claims 1 to 34 or a pharmaceutical composition as defined in claim 35 for use in wound care.

59. Use of a composition as defined in any one of claims 1 to 34 or a pharmaceutical composition as defined in claim 35 in the manufacture of a medicament for the treatment of microbial infections.

60. Use of a composition as defined in any one of claims 1 to 34 or a pharmaceutical composition as defined in claim 35 in the manufacture of a medicament for the treatment of wounds.

61. A method of treating an individual with a microbial infection, the method comprising the step of administering to an individual in need thereof an effective amount of a composition as defined in any one of claims 1 to 34 or pharmaceutical composition as defined in claim 35.

62. A method of treating a wound in an individual, the method comprising the step of administering to an individual in need thereof an effective amount of a composition as defined in any one of claims 1 to 34 or a pharmaceutical composition as defined in claim 35.

63. A method for killing microorganisms in vitro comprising contacting the microorganisms with a composition as described in any one of claims 1 to 34 or a pharmaceutical composition as defined in claim 35.

64. A composition or pharmaceutical composition for use according to claims 50 to 58, a use according to claim 59 or 60, or a method according to claims 61 to 63, wherein the composition or pharmaceutical composition is coated or impregnated onto, or admixed or otherwise associated with, a medical device, implant, wound care product, or material for use in the same.

65. A composition substantially as described herein with reference to the description and figures.

66. A medical implant or device, or biomaterial for use in the same, substantially as described herein with reference to the description and figures.

67. Use of a composition substantially as described herein with reference to the description and figures.

68. A method for treating or preventing infection substantially as described herein with reference to the description and figures.

69. A method for treating wounds substantially as described herein with reference to the description and figures.


SEQUENCE LISTING (24 sequences)

SEQ ID NO: 1 - protein, 28 aa, Artificial Sequence
  GVR28 peptide
GVRPDGFAHIRDFVSRIVRRLNIGPSKV

SEQ ID NO: 2 - protein, 25 aa, Artificial Sequence
  FYL25 peptide
FYLKTYRSQAPVLDAIRRLRLRGGS

SEQ ID NO: 3 - protein, 25 aa, Artificial Sequence
  FFL25 peptide
FFLKDFSTKRQIIDAINKVVYKGGR

SEQ ID NO: 4 - protein, 30 aa, Artificial Sequence
  VTT30 peptide
VTTEIRFADSKRKSVLLDKIKNLQVALTSK

SEQ ID NO: 5 - protein, 33 aa, Artificial Sequence
  SFV33 peptide
SFVARNTFKRVRNGFLMRKVAVFFSNTPTRASP

SEQ ID NO: 6 - protein, 32 aa, Artificial Sequence
  DVN32 peptide
DVNVFAIGVEDADEGALKEIASEPLNMHMFNL

SEQ ID NO: 7 - protein, 20 aa, Artificial Sequence
  KPE20 peptide
KPEILNLVKRMKIKTGKALN

SEQ ID NO: 8 - protein, 20 aa, Artificial Sequence
  GFA20 peptide
GFAHIRDFVSRIVRRLNIGP

SEQ ID NO: 9 - protein, 20 aa, Artificial Sequence
  QAP20 peptide
QAPVLDAIRRLRLRGGSPLN

SEQ ID NO: 10 - protein, 20 aa, Artificial Sequence
  KGF20 peptide
KGFESKVDAILNRISQMHRV

SEQ ID NO: 11 - protein, 20 aa, Artificial Sequence
  RKV20 peptide
RKVAVFFSNTPTRASPQLRE

SEQ ID NO: 12 - protein, 20 aa, Artificial Sequence
  VAA20 peptide
VAAKPVATKMAVRPPVAVKP

SEQ ID NO: 13 - protein, 20 aa, Artificial Sequence
  AAK20 peptide
AAKPVATKPEVPRPQAAKPA

SEQ ID NO: 14 - protein, 20 aa, Artificial Sequence
  TTK20 peptide
TTKPVTTTKPVTTTTKPVTT

SEQ ID NO: 15 - protein, 76 aa, Artificial Sequence
  AAA76 peptide
AAAKPAPAKPVAAKPVATKMATVRPPVAVKPATAAKPVAAKPAAVRPPAAAAAKPVATKPEVPRPQAAKPAATKPA

SEQ ID NO: 16 - protein, 36 aa, Artificial Sequence
  TSS36 peptide
TSSPTSNPVTTTKPVTTTKPVTTTTKPVTTTTKPVT

SEQ ID NO: 17 - protein, 20 aa, Artificial Sequence
  YDR20 peptide
YDRLIKESRRQKTRVFAVVI

SEQ ID NO: 18 - protein, 20 aa, Artificial Sequence
  EQN20 peptide
EQNFHKARRFVEQVARRLTL

SEQ ID NO: 19 - protein, 20 aa, Artificial Sequence
  VVH20 peptide
VVHAINAIVRSPRGGARRHA

SEQ ID NO: 20 - protein, 20 aa, Artificial Sequence
  LRL20 peptide
LRLKPYGALVDKVKSFTKRF

SEQ ID NO: 21 - protein, 20 aa, Artificial Sequence
  FTK20 peptide
FTKRFIDNLRDRYYRCDRNL

SEQ ID NO: 22 - protein, 20 aa, Artificial Sequence
  RDA20 peptide
RDALKSSVDAVKYFGKGTYT

SEQ ID NO: 23 - protein, 20 aa, Artificial Sequence
  TKR20 peptide
TKRFAKRLAERFLTAGRTDP

SEQ ID NO: 24 - protein, 37 aa, Artificial Sequence
  LL37 peptide
LLGDFFRKSKEKIGKEFKRIVQRIKDFLRNLVPRTES